(19) United States
(12) Patent Application Publication
(10) Pub. No.: US 2003/0069285 A1
(43) Pub. Date: Apr. 10, 2003

(54) Novel solvate and crystalline forms of lercanidipine hydrochloride

(75) Inventors: Amedeo Leonardi, Milan (IT); Gianluca De Iasi, Aprilia (IT); Fausto Bonifacio, Latina (IT)

Correspondence Address: Mitchell Bernstein, Ph.D., DARBY & DARBY P.C., Post Office 5257, New York, NY, 10150-5257, US

(73) Assignee: Recordati Ireland Limited

(21) Appl. No.: 10/214,385

(22) Filed: Aug. 6, 2002

Related U.S. Application Data
(60) Provisional application No. 60/367,789, filed on Mar. 26, 2002.

(30) Foreign Application Priority Data
Aug. 6, 2001 (IT) MI 2001A 001726
Aug. 6, 2001 (IT) MI 2001A001727

(51) Int. Cl. C07D 213/46; A61K 31/44

(52) U.S. Cl. 514/355; 546/315

(57) ABSTRACT

The invention describes new solvates of lercanidipine hydrochloride with organic solvents, new lercanidipine hydrochloride crystalline Forms (III) and (IV) obtained from said solvates, pharmaceutical and antihypertensive compositions containing as active agent at least one of the lercanidipine hydrochloride crystalline Forms (III) and (IV) and methods of use thereof.

### RELATED APPLICATIONS

The present application claims priority under 35 U.S.C. 119 (e) of U.S. provisional application No. 60/367,789, filed Mar. 26, 2002 and priority under 35 U.S.C. 119 (a)-(d) of Italian patent applications MI 2001A 001726 and MI 2001A 001727, each filed Aug. 6, 2001. Each of the aforementioned applications is hereby incorporated herein by reference in its entirety.

### FIELD OF THE INVENTION

The invention describes novel solvate forms and crystalline forms of lercanidipine hydrochloride, and processes for the preparation of these forms. Pharmaceutical compositions comprising the novel crystalline forms are also contemplated.

### BACKGROUND OF THE INVENTION

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist with long duration of action and high vascular selectivity. Its mechanism of antihypertensive activity is attributed to a direct relaxant effect on vascular smooth muscle, which lowers total peripheral resistance. The recommended starting dose of lercanidipine hydrochloride as monotherapy is 10 mg daily by oral route, with a drug titration to 20 mg daily. Lercanidipine is rapidly absorbed following oral administration with peak plasma levels occurring 2-3 hours following dosing. Elimination is essentially via the hepatic route.

By virtue of its high lipophilicity and high membrane coefficient, lercanidipine combines a short plasma half life with a long duration of action. In fact, the preferential distribution of the drug into membranes of smooth muscle cells results in membrane-controlled pharmacokinetics characterized by a prolonged pharmacological effect. In comparison to other calcium antagonists, lercanidipine is characterized by gradual onset and long-lasting duration of action despite decreasing plasma levels. In vitro studies show that isolated rat aorta response to high K⁺ may be attenuated by lercanidipine, even after the drug has been removed from the environment of the aortic tissue for 6 hours.

Lercanidipine is commercially available from Recordati S.p.A. (Milan, Italy) and has been described along with methods for making it and resolving it into individual enantiomers in U.S. Pat. Nos. 4,705,797; 5,767,136; 4,968,832; 5,912,351; and 5,696,139.

A process for preparing lercanidipine described in U.S. Pat. No. 4,705,797 involves the following scheme:

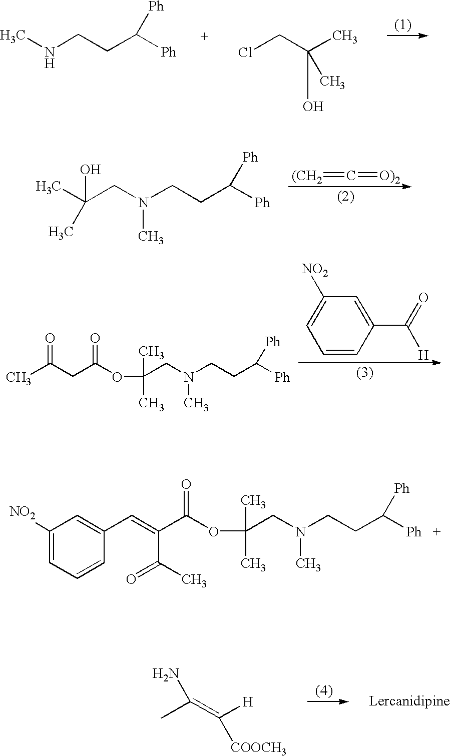

(1):xylene at reflux; (2):toluene, 85° C.; (3) HCl+CHCl₃; 0° C.; (4) HO—CH(CH₃)₂ at reflux

Crude lercanidipine is an oily residue that may be purified by flash chromatography using chloroform, containing increasing amounts of acetone, as the eluent. The solvent is then evaporated to dryness and remaining residue is dissolved in methanol adding a small excess of hydrochloric acid in ethanol. After evaporation of the solvent, the hemi-hydrated hydrochloride salt is prepared by treatment with diluted hydrochloric acid in the presence of sodium chloride.

A major disadvantage of the process of preparing lercanidipine, as it is described in U.S. Pat. No. 4,705,797, is that the disclosed cyclization reaction generates several by-products, which results in a lower yield for the desired product. Moreover, the purification and isolation of lercanidipine from the reaction mixture is quite complex, since it requires numerous treatments with different solvents. Finally, the purification and isolation steps are difficult to perform on an industrial scale because of the necessity of purifying the product by column chromatography.

U.S. Pat. No. 5,912,351 describes a simpler process for the preparation of lercanidipine hydrochloride. It involves reaction of 1,4-dihydro-2,6-dimethyl-5-methoxycarbonyl-4-(3-nitrophenyl) pyridine-3-carboxylic acid with thionyl chloride in dichloromethane and dimethylformamide at a temperature between −4 and +1° C. and subsequent esterification of the obtained acid chloride with 2, N-dimethyl-N-(3,3-diphenylpropyl)-1-amino-2-propyl alcohol at a temperature between −10 and 0° C. The process yields lercanidipine hydrochloride in an anhydrous non-hygroscopic crystalline form, and avoids the formation of unwanted by-products and the subsequent purification on chromatography columns.

However, the isolation of lercanidipine hydrochloride in crystalline form is again quite complex. After evaporating solvents from the reaction mixture and dissolving the residue thus obtained in ethyl acetate, the solution is washed first with brine, then washed further sequentially five times with a 10% solution of sodium carbonate, five times with 1N hydrochloric acid and finally once again with brine.

Therefore, there is a need in the art for a process for the preparation of lercanidipine hydrochloride in crystalline form which does not have any of the disadvantages of the currently used processes.

In addition, it was observed that lercanidipine, as produced by the second-described process above, displayed batch-to-batch variability despite careful process control and even observation of the melting point of 186-188° C. believed to be characteristic of the solid product obtained by the process of Example 3 of U.S. Pat. No. 5,767,136. This variability was manifest in seemingly unpredictably appearing (and disappearing) differences in one or more of product appearance (e.g., color), melting point and solubility. This raised issues as to whether assurances of purity and/or reproducibility can be made (e.g., to regulatory authorities) that the product is always the same.

Further research by the present inventors revealed batch-to-batch differences in bioavailability in animals, and differences in crystal size. In the course of researching the causes of the variability problem, the inventors surprisingly discovered novel lercanidipine solvates polymorphs. They also discovered more suitable processes for the preparation and isolation of crystalline lercanidipine hydrochloride products from the reaction mixture. It was surprisingly determined that lercanidipine hydrochloride shows polymorphic features and crystallizes into different novel crystalline forms depending on the process followed and on the solvents used. Furthermore, the isolation of each of these individual crystalline polymorphs has become possible, thus decreasing the possibility of batch to batch variability of lercanidipine, which the present inventors determined was due to mixtures of different solid forms being present by the same batch and to such mixtures of different composition having a melting point within the same range as individual crystalline forms. As a result, more reproducible batches of lercanidipine hydrochloride more suitable for large scale manufacture and quality control became available by the present inventors.

Accordingly, herein Applicants disclose two additional novel crystalline forms which are formed from novel solvate forms of lercanidipine.

### SUMMARY OF THE INVENTION

The present invention provides novel solvates and crystalline forms of lercanidipine hydrochloride and processes for making them.

In certain embodiments, the invention provides solvates of lercanidipine hydrochloride comprising lercanidipine hydrochloride and an organic solvent. In preferred embodiments, the solvent is selected from the group consisting of methylene chloride, acetone, anisole, tetrahydrofuran, terbutyl methyl ether, isopropanol, 2-butanol, heptane, methyl ethyl ketone, and ethyl acetate.

In one embodiment, the invention provides a solvate of lercanidipine hydrochloride wherein the solvent is methylene chloride, the lercanidipine hydrochloride-methylene chloride content is 1:1 (mole/mole), and the solvate has, at wavelength Kα, an X-ray diffraction image expressed by the following Table:

| d (Å) | Relative intensity (%) | Angle (°2 θ) |
|---|---|---|
| 6.6 | 40 | 13.4 |
| 5.87 | 42 | 15.1 |
| 5.04 | 39 | 17.6 |
| 4.00 | 96 | 22.2 |
| 3.90 | 29 | 22.8 |
| 3.86 | 34 | 23.0 |
| 3.67 | 100 | 24.2 |
| 2.04 | 31 | 44.4 |

In another embodiment the invention provides a solvate of lercanidipine hydrochloride wherein the solvent is anisole, the lercanidipine hydrochloride-anisole content is 1:0.4 (mole/mole), and the solvate (a) form has, at wavelength Kα, an X-ray diffraction image expressed by the following Table:

| d (Å) | Relative intensity (%) | Angle (°2 θ) |
|---|---|---|
| 17.4 | 62 | 5.1 |
| 7.6 | 34 | 11.6 |
| 5.71 | 43 | 15.5 |
| 5.57 | 58 | 15.9 |
| 4.99 | 47 | 17.7 |
| 4.62 | 40 | 19.2 |
| 4.44 | 29 | 20.0 |
| 4.28 | 98 | 20.8 |
| 4.04 | 100 | 22.0 |
| 3.19 | 43 | 27.9 |
| 2.92 | 36 | 30.6 |
| 2.86 | 42 | 31.3 |

In another embodiment the invention provides a solvate of lercanidipine hydrochloride wherein the solvent is anisole, the lercanidipine hydrochloride-anisole content is 1:0.4 (mole/mole), and the solvate (b) form has, at wavelength Kα, an X-ray diffraction image expressed by the following Table:

| d (Å) | Relative intensity (%) | Angle (°2 θ) |
|---|---|---|
| 6.9 | 49 | 12.8 |
| 6.7 | 63 | 13.3 |
| 5.82 | 86 | 15.2 |
| 5.27 | 41 | 16.8 |
| 5.15 | 53 | 17.2 |
| 4.00 | 47 | 22.2 |
| 3.89 | 46 | 22.8 |
| 3.66 | 100 | 24.3 |

In another embodiment the invention provides a solvate of lercanidipine hydrochloride wherein the solvent is acetone, the lercanidipine hydrochloride-acetone content is 1:1.2 (mole/mole), and the solvate has, at wavelength Kα, an X-ray diffraction image expressed by the following Table:

| d (Å) | Relative intensity (%) | Angle (°2 θ) |
|---|---|---|
| 10.1 | 42 | 8.8 |
| 7.3 | 100 | 12.1 |
| 5.87 | 31 | 15.1 |
| 4.07 | 41 | 21.8 |
| 3.96 | 52 | 22.4 |
| 3.79 | 49 | 23.5 |
| 3.71 | 37 | 24.0 |
| 3.34 | 33 | 26.7 |

In another embodiment the invention provides a solvate of lercanidipine hydrochloride wherein the solvent is ethyl acetate, the lercanidipine hydrochloride-ethyl acetate content is 1:1 (mole/mole), and the solvate has, at wavelength Kα, an X-ray diffraction image expressed by the following Table:

| D (Å) | Relative intensity (%) | Angle (°2 θ) |
|---|---|---|
| 6.9 | 100 | 12.8 |
| 6.3 | 29 | 14.0 |
| 5.80 | 45 | 15.3 |
| 5.65 | 31 | 15.7 |
| 5.43 | 44 | 16.3 |
| 4.74 | 53 | 18.7 |
| 4.53 | 49 | 19.6 |
| 4.00 | 84 | 22.2 |
| 3.91 | 91 | 22.7 |
| 3.67 | 77 | 24.2 |
| 3.60 | 34 | 24.7 |
| 3.53 | 34 | 25.2 |
| 3.49 | 43 | 25.5 |

In another embodiment the invention provides a solvate of lercanidipine hydrochloride wherein the solvent is terbutyl methyl ether, the lercanidipine hydrochloride-terbutyl methyl ether content is 1:0.8 (mole/mole), and the solvate has, at wavelength Kα, an X-ray diffraction image expressed by the following Table:

| d (Å) | Relative intensity (%) | Angle (°2 θ) |
|---|---|---|
| 6.2 | 77 | 14.2 |
| 4.88 | 29 | 18.2 |
| 4.52 | 64 | 19.6 |
| 4.02 | 48 | 22.1 |
| 3.93 | 100 | 22.6 |
| 3.43 | 46 | 26.0 |

In another embodiment the invention provides a solvate of lercanidipine hydrochloride wherein the solvent is isopropanol, the lercanidipine hydrochloride-isopropanol content is 1:1 (mole/mole), and the solvate has, at wavelength Kα, an X-ray diffraction image expressed by the following Table:

| d (Å) | Relative intensity (%) | Angle (°2 θ) |
|---|---|---|
| 6.6 | 35 | 13.5 |
| 5.85 | 48 | 15.1 |
| 5.06 | 41 | 17.5 |
| 4.04 | 64 | 22.0 |
| 3.90 | 39 | 22.8 |
| 3.72 | 37 | 23.9 |
| 3.67 | 100 | 24.2 |

In another embodiment the invention provides a solvate of lercanidipine hydrochloride wherein the solvent is 2-butanol, the lercanidipine hydrochloride-2-butanol content is 1:0.8 (mole/mole), and the solvate has, at wavelength Kα, an X-ray diffraction image expressed by the following Table:

| d (Å) | Relative intensity (%) | Angle (°2 θ) |
|---|---|---|
| 6.8 | 34 | 13.1 |
| 5.86 | 36 | 15.1 |
| 5.13 | 42 | 17.3 |
| 4.03 | 51 | 22.0 |
| 3.90 | 36 | 22.8 |
| 3.67 | 100 | 24.2 |

In another embodiment the invention provides a solvate of lercanidipine hydrochloride wherein the solvent is heptane, the lercanidipine hydrochloride-heptane content is 1:0.9 (mole/mole), and the solvate has, at wavelength Kα, an X-ray diffraction image expressed by the following Table:

| d (Å) | Relative intensity (%) | Angle (°2 θ) |
|---|---|---|
| 7.3 | 54 | 12.2 |
| 6.0 | 44 | 14.7 |
| 4.03 | 85 | 22.0 |
| 3.85 | 100 | 23.1 |
| 3.76 | 93 | 23.6 |
| 3.63 | 67 | 24.5 |
| 3.38 | 39 | 26.4 |
| 3.01 | 47 | 29.6 |

In another embodiment the invention provides a solvate of lercanidipine hydrochloride wherein the solvent is methyl ethyl ketone, the lercanidipine hydrochloride-methyl ethyl ketone content is 1:0.7 (mole/mole), and the solvate has, at wavelength Kα, an X-ray diffraction image expressed by the following Table:

| d (Å) | Relative intensity (%) | Angle (°2 θ) |
|---|---|---|
| 6.8 | 50 | 13.1 |
| 6.1 | 43 | 14.5 |
| 5.87 | 47 | 15.1 |
| 5.10 | 53 | 17.4 |
| 3.99 | 100 | 22.2 |
| 3.87 | 48 | 22.9 |
| 3.74 | 36 | 23.8 |
| 3.69 | 65 | 24.1 |
| 3.61 | 70 | 24.6 |

In another embodiment the invention provides a solvate of lercanidipine hydrochloride wherein the solvent is tetrahydrofuran, the lercanidipine hydrochloride-tetrahydrofuran content is 1:0.9 (mole/mole), and the solvate has, at wavelength Kα, an X-ray diffraction image expressed by the following Table:

| d (Å) | Relative intensity (%) | Angle (°2 θ) |
|---|---|---|
| 6.6 | 100 | 13.5 |
| 5.88 | 32 | 15.1 |
| 5.12 | 56 | 17.3 |
| 4.25 | 38 | 20.9 |
| 4.06 | 50 | 21.9 |
| 3.92 | 42 | 22.7 |
| 3.75 | 44 | 23.7 |
| 3.70 | 90 | 24.0 |
| 3.64 | 31 | 24.4 |

In yet another embodiment, the invention provides isolated lercanidipine hydrochloride crystalline form (III), having a melting point in the range of 137-150° C. and having an X-ray diffraction image, at wavelength Kα, expressed by the following Table:

| d (Å) | Relative intensity (%) | Angle (°2 θ) |
|---|---|---|
| 11.5 | 39 | 7.7 |
| 9.1 | 38 | 9.7 |
| 9.0 | 37 | 9.8 |
| 8.0 | 50 | 11.0 |
| 6.6 | 48 | 13.5 |
| 5.58 | 57 | 15.9 |
| 5.49 | 34 | 16.1 |
| 5.13 | 43 | 17.3 |
| 4.09 | 63 | 21.7 |
| 3.92 | 43 | 22.7 |
| 3.72 | 100 | 23.9 |
| 3.60 | 85 | 24.7 |
| 3.47 | 31 | 25.6 |

In another embodiment, the invention provides crystalline form (IV), having a melting point in the range of 116-135° C. and having an X-ray diffraction image, at wavelength Kα, expressed by the following Table:

| d (Å) | Relative intensity (%) | Angle (°2 θ) |
|---|---|---|
| 7.9 | 71 | 11.2 |
| 6.9 | 53 | 12.7 |
| 5.21 | 57 | 17.0 |
| 5.13 | 46 | 17.3 |
| 4.73 | 66 | 18.8 |
| 4.69 | 95 | 18.9 |
| 4.53 | 53 | 19.6 |
| 4.40 | 81 | 20.2 |
| 4.34 | 43 | 20.4 |
| 3.99 | 44 | 22.2 |
| 3.89 | 52 | 22.8 |
| 3.77 | 100 | 23.6 |
| 3.69 | 35 | 24.1 |

Also provided are pharmaceutical compositions comprising (1) lercanidipine hydrochloride crystalline Form (m) or lercanidipine hydrochloride crystalline Form (IV), and combinations thereof, comprising a predetermined content of each crystalline form, and optionally including other forms of lercanidipine, such as, amorphous lercanidipine, lercanidipine hydrochloride crystalline Form (I) or lercanidipine hydrochloride crystalline Form (II), and (2) at least one component selected from the group consisting of a pharmaceutically acceptable carrier or diluent, a flavorant, a sweetener, a preservative, a dye, a binder, a suspending agent, a dispersing agent, a colorant, a disintegrant, an excipient, a lubricant, a plasticizer, and an edible oil.

In certain embodiments the aforementioned pharmaceutical compositions are provided as a dosage form comprising lercanidipine hydrochloride crystalline Form (III) or Form (IV) or a combination thereof having a predetermined formulation of each crystalline Form, optionally including other forms of lercanidipine, such those set forth above.

In further embodiments, the invention provides lercanidipine hydrochloride crystalline forms (III) or (IV) or mixtures thereof, including the dosage forms set forth above, wherein said crystalline forms are in micronized form, preferably with an average size of D(50%) 2-8 μm, D(90%)<15 μm.

In another embodiment, a method is provided for treating a subject with arterial hypertension, the method comprising administering a therapeutically effective amount of lercanidipine hydrochloride crystalline Form (III), lercanidipine hydrochloride crystalline form (IV), or combinations thereof to a subject in need of such treatment.

In another embodiment, the invention provides a method of treating or preventing atherosclerotic lesions in arteries in a subject, which comprises administering a therapeutically effective amount of lercanidipine hydrochloride crystalline Form (III), lercanidipine hydrochloride crystalline Form (IV), or combinations thereof having a predetermined content in each of said Form (III) and (IV) to a subject in need of such treatment.

In other embodiments, the invention provides an antihypertensive composition comprising predetermined amounts of lercanidipine hydrochloride crystalline Form (III) and lercanidipine hydrochloride crystalline Form (IV). In certain embodiments, the ratio of Form (III):Form (IV) is between about 1:9 to 9:1. e.g., wherein the ratio of Form (III):Form (IV) is selected from the group consisting of 9:1, 7:3, 1:1, 3:7 and 1:9.

Also provided are methods of making a lercanidipine hydrochloride-methylene chloride solvate or a lercanidipine hydrochloride-methyl ethyl ketone solvate from the starting material of lercanidipine hydrochloride crystalline Form I, methods for preparing lercanidipine hydrochloride crystalline Form (III) by the removal of solvent from a lercanidipine hydrochloride solvate by evaporation under vacuum or in nitrogen stream to form said crystalline Form (III) and a method for preparing lercanidipine hydrochloride crystalline Form (IV) by removal of acetone from a lercanidipine hydrochloride-acetone solvate by evaporation under vacuum or in a nitrogen stream.

These and other aspects of the invention will be apparent to those of ordinary skill in the art in light of the present description, claims and figures.

### BRIEF DESCRIPTION OF DRAWINGS

FIG. 1 shows the X-ray diffraction spectrum at wavelength Kα of the solvate of lercanidipine hydrochloride with methylene chloride having a lercanidipine hydrochloride-methylene chloride content of 1:1 (mole/mole). The ordinate indicates the number of counts per second and the abscissa represents the values of 2θ angles.

FIG. 2 shows the X-ray diffraction spectrum at wavelength Kα of crystalline form (III) of lercanidipine hydrochloride.

FIGS. 3 and 4 show plots referring to the solvate of lercanidipine hydrochloride with methylene chloride having a lercanidipine hydrochloride-methylene chloride content of 1:1 (mole/mole)and of lercanidipine hydrochloride crystalline form (III) and the thermogravimetric analysis carried out according to the operating modes described in Example 36B. The ordinate indicates % mass variation and the abscissa the temperature.

FIGS. 5 and 6 show Raman spectrums referring to the solvate of lercanidipine hydrochloride with methylene chloride having a lercanidipine hydrochloride-methylene chloride content of 1:1 (mole/mole) and of lercanidipine hydrochloride crystalline form (III), respectively. The ordinate indicates Raman units and the abscissa represents wave number (in cm⁻¹).

FIG. 7 shows the X-ray diffraction spectrum of lercanidipine hydrochloride crystalline form (IV).

FIG. 8 shows the X-ray diffraction spectrum of the solvate lercanidipine hydrochloride-acetone having a lercanidipine hydrochloride-acetone content of 1:1.2(mole/mole).

FIG. 9 shows the X-ray diffraction spectrum of the solvate lercanidipine hydrochloride-ethyl acetate having a lercanidipine hydrochloride-ethyl acetate content of 1:1 (mole/mole).

FIG. 10 shows the X-ray diffraction spectrum of the solvate lercanidipine hydrochloride-tetrahydrofuran having a lercanidipine hydrochloride-tetrahydrofuran content of 1:0.9(mole/mole).

FIG. 11 shows the X-ray diffraction spectrum of the solvate lercanidipine hydrochloride-terbutyl methyl ether having a lercanidipine hydrochloride-terbutyl methyl ether content of 1:0.8 (mole/mole).

FIG. 12 shows the X-ray diffraction spectrum of the solvate lercanidipine hydrochloride-anisole (a) form having a lercanidipine hydrochloride-anisole content of 1:0.4 (mole/mole).

FIG. 13 shows the X-ray diffraction spectrum of the solvate lercanidipine hydrochloride-anisole (b) form having a lercanidipine hydrochloride-anisole content of 1:0.4 (mole/mole).

FIG. 14 shows the X-ray diffraction spectrum of the solvate lercanidipine hydrochloride-isopropanol having a lercanidipine hydrochloride-isopropanol content of 1:1 (mole/mole).

FIG. 15 shows the X-ray diffraction spectrum of the solvate lercanidipine hydrochloride-isobutanol having a lercanidipine hydrochloride-isobutanol content of 1:0.8 (mole/mole).

FIG. 16 shows the X-ray diffraction spectrum of the solvate lercanidipine hydrochloride-heptane having a lercanidipine hydrochloride-heptane content of 1:0.9 (mole/mole).

FIG. 17 shows the Raman spectrum of lercanidipine hydrochloride crystalline form (IV).

FIG. 18 shows the Raman spectrum of the solvate lercanidipine hydrochloride-acetone having a lercanidipine hydrochloride-acetone content of 1:1.2(mole/mole).

FIG. 19 shows the Raman spectrum of the solvate lercanidipine hydrochloride-ethyl acetate having a lercanidipine hydrochloride-ethyl acetate content of 1:1 (mole/mole).

FIG. 20 shows the Raman spectrum of the solvate lercanidipine hydrochloride-tetrahydrofuran having a lercanidipine hydrochloride-tetrahydrofuran content of 1:0.9 (mole/mole).

FIG. 21 shows the Raman spectrum of the solvate lercanidipine hydrochloride-terbutyl methyl ether having a lercanidipine hydrochloride-terbutyl methyl ether content of 1:0.8 (mole/mole).

FIG. 22 shows the Raman spectrum of the solvate lercanidipine hydrochloride-anisole (a) form having a lercanidipine hydrochloride-anisole content of 1:0.4(mole/mole).

FIG. 23 shows the Raman spectrum of the solvate lercanidipine hydrochloride-anisole (b) form having a lercanidipine hydrochloride-anisole content of 1:0.4(mole/mole).

FIG. 24 shows the Raman spectrum of the solvate lercanidipine hydrochloride-isopropanol having a lercanidipine hydrochloride-isopropanol content of 1:1 (mole/mole).

FIG. 25 shows the Raman spectrum of the solvate lercanidipine hydrochloride-isobutanol having a lercanidipine hydrochloride-isobutanol content of 1:0.8 (mole/mole).

FIG. 26 shows the Raman spectrum of the solvate lercanidipine hydrochloride-heptane having a lercanidipine hydrochloride-heptane content of 1:0.9 (mole/mole).

FIG. 27 shows the results of the thermogravimetric analysis carried out on the solvate lercanidipine hydrochloride-anisole (b) form having a lercanidipine hydrochloride-anisole content of 1:0.4 (mole/mole).

FIG. 28 shows the results of the thermogravimetric analysis carried out on the solvate lercanidipine hydrochloride-ethyl acetate having a lercanidipine hydrochloride-ethyl acetate content of 1:1 (mole/mole).

FIG. 29 shows the results of the thermogravimetric analysis carried out on the solvate lercanidipine hydrochloride-acetone having a lercanidipine hydrochloride-acetone content of 1:1.2 (mole/mole).

FIG. 30 shows the results of the thermogravimetric analysis carried out on the solvate lercanidipine hydrochloride-tetrahydrofuran having a lercanidipine hydrochloride-tetrahydrofuran content of 1:0.9 (mole/mole).

FIG. 31 shows the results of the thermogravimetric analysis carried out on the solvate lercanidipine hydrochloride-anisole (a) form having a lercanidipine hydrochloride-anisole content of 1:0.4 (mole/mole).

FIG. 32 shows the results of the thermogravimetric analysis carried out on the solvate lercanidipine hydrochloride-terbutyl methyl ether having a lercanidipine hydrochloride-terbutyl methyl ether content of 1:0.8 (mole/mole).

FIG. 33 shows the results of the thermogravimetric analysis carried out on the solvate lercanidipine hydrochloride-isopropanol having a lercanidipine hydrochloride-isopropanol content of 1:1 (mole/mole).

FIG. 34 shows the results of the thermogravimetric analysis carried out on the solvate lercanidipine hydrochloride-isobutanol having a lercanidipine hydrochloride-isobutanol content of 1:0.8 (mole/mole).

FIG. 35 shows the results of the thermogravimetric analysis carried out on the solvate lercanidipine hydrochloride-heptane having a lercanidipine hydrochloride-heptane content of 1:0.9 (mole/mole).

FIG. 36 shows the results of the thermogravimetric analysis carried out on lercanidipine hydrochloride crystalline form (IV).

FIG. 37 shows the results of the thermogravimetric analysis carried out on the solvate lercanidipine hydrochloride-methyl ethyl ketone having a lercanidipine hydrochloride-methyl ethyl ketone content of 1:0.7 (mole/mole).

FIG. 38 shows the X-ray spectrum of the solvate lercanidipine hydrochloride-methyl ethyl ketone having a lercanidipine hydrochloride-methyl ethyl ketone content of 1:0.7 (mole/mole).

FIG. 39 shows the Raman spectrum of the solvate lercanidipine hydrochloride-methyl ethyl ketone having a lercanidipine hydrochloride-methyl ethyl ketone content of 1:0.7 (mole/mole).

### DETAILED DESCRIPTION OF THE INVENTION

The present invention discloses novel solvates of lercanidipine hydrochloride and processes for making them. Applicants also have determined that removal of the solvents from the solvates discussed herein under specific conditions produces novel crystalline forms of lercanidipine hydrochloride. Specifically, these forms are termed crystalline Forms (III) and (IV).

The novel methods for preparation of solvates of lercanidipine hydrochloride described herein are suitable for highly reproducible commercial scale production of reproducible solid compositions of lercanidipine hydrochloride. The methods advantageously produce novel solvates and crystalline forms starting with crude Forms (A) or (B) of lercanidipine hydrochloride or crystalline Forms (I) and (II) that are described in Italian patent application no. MI 2001A 001726, filed Aug. 6, 2001, U.S. provisional application No. 60/367,789, filed Mar. 26, 2002, and co-pending U.S. patent application Ser. No. ______ of Bonifacio et al., for “NOVEL CRUDE AND CRYSTALLINE FORMS OF LERCANIDIPINE HYDROCHLORIDE” (Docket no.: 4266/1J959-US1), filed Aug. 6, 2002, and which exhibit characteristics desirable for industrial applications. Crude Forms (A) and (B), e.g., exhibit higher solubility and faster drying rates compared to other crude forms of lercanidipine hydrochloride that have previously been reported. Hence, it is desirable to produce crystalline forms of lercanidipine hydrochloride using crude Form (A) or (B) as starting material. The solvates and crystalline forms of the present invention may also be produced using other forms of lercanidine, such as, for example and without limitation, amorphous lercanidipine and lercanidipine crude Form (C).

Methods known in the art for producing crystalline lercanidipine hydrochloride were inconsistent in producing lercanidipine hydrochloride with predictable physical and chemical characteristics. Hence, prior art methods had the undesirable property of producing lercanidipine hydrochloride that varied, e.g., in physico-chemical properties, from batch to batch, even among batches produced by the same process and under the same conditions. The present inventors have discovered that the source of inconsistency exhibited by the prior art methods of producing lercanidipine hydrochloride is the presence of varying and unpredictable amounts of crystalline lercanidipine hydrochloride Form (II). In contrast to prior art methods of producing lercanidipine hydrochloride, the invention provides the novel crystalline Forms (III) and (IV) that represent crystalline forms of lercanidipine hydrochloride that has not been obtained with previously achieved solid forms of lercanidipine hydrochloride.

As used herein, the term “crude form” refers to precipitated solid forms comprising crystals of a compound that have not been washed and/or recrystallized to remove impurities (including but not limited to solvent) that may be present and which lack melting point and x-ray spectra characteristic of crystalline forms. In the present specification, the crude forms are referred to as Forms (A) and (B) and (C) of lercanidipine hydrochloride.

As used herein, the term “crystalline form” refers to crystals of a compound that have been crystallized and treated to remove impurities, e.g., a form obtained after evaporation of solvent from a solvate, or having melting point and x-ray spectra characteristic of crystalline forms. Crystalline Forms (I) and (II) of lercanidipine hydrochloride are described in Italian patent application no. MI 2001A 001726, filed Aug. 6, 2001, U.S. provisional application No. 60/367,789, filed Mar. 26, 2002, and co-pending U.S. patent application Ser. No. ______ of Bonifacio et al., for “NOVEL CRUDE AND CRYSTALLINE FORMS OF LERCANIDIPINE HYDROCHLORIDE,” (Docket no.:4266/1J959-US1), filed Aug. 6, 2002. Lercanidipine hydrochloride crystalline Forms (III) and (IV) are described by their X-ray structure, Raman spectra and melting points, which are discussed below. Alternatively, these crystalline forms can be described by a process that yields them, e.g., removal of solvent from a solvate under specified conditions. These crystalline forms have an HPLC purity of >99% and residual solvents content of ≦3000 ppm.

As used herein, the term “polymorphic” or “polymorphism” refers to a property of a compound to exist in two or more forms with distinct structures. The different crystalline forms can be detected by crystallographic techniques or indirectly by assessment of differences in physical and/or chemical properties associated with each particular polymorph. Within this application, crystalline Forms (III) and (IV) are considered polymorphs of lercanidipine hydrochloride.

The term “solvate” refers to an aggregate that consists of a solvate ion or molecule in complex with 1.2 or less solvent molecules, on a mol/mol basis. Within this application, solvates are written as “molecule-solvent.” In other words, a hyphen is used to separate the molecule and solvent that produce the whole solvate. Therefore, the phrase “lercanidipine hydrochloride-acetone” refers to a solvate where the molecule is lercanidipine hydrochloride and the solvent is acetone.

As used herein, a “subject in need of treatment” is a mammalian (e.g., human) subject suffering from or at risk of developing the particular condition to be treated, e.g., essential hypertension, secondary hypertension, isolated systolic hypertension, coronary heart disease (e.g., chronic stable angina, myocardial infarction), congestive heart failure. A subject in need of treatment for arterial hypertension may be identified using methods well known in the art such as, for example, by direct measurement of blood pressure using, for example, a manual sphygmomanometer, automatic/electronic devices or ambulatory blood pressure monitoring.

As used herein, a “therapeutically effective amount” of an agent is an amount sufficient to ameliorate at least one symptom associated with a pathological, abnormal or otherwise undesirable condition, an amount sufficient to prevent or lessen the probability that such a condition will occur or re-occur, or an amount sufficient to delay worsening of such a condition. An amount sufficient to lower blood pressure to values lower than 140/90 is recommended. Recent World Health Organization guidelines recommended a diastolic blood pressure lower than 85 mm Hg and a systolic blood pressure lower than 130 mm Hg in younger patients and in diabetic patients. Treatment of other pathologies, such as, for example, heart failure or atherosclerosis is also specifically contemplated as per, e.g., U.S. Pat. Nos. 5,696,139 and 5,767,136.

Subjects suffering from and in need of treatment of hypertension and the other conditions mentioned above can be treated by the administering a therapeutically effective amount of isolated lercanidipine hydrochloride crystalline Form (III), lercanidipine hydrochloride crystalline Form (IV), or combinations thereof, of predetermined polymorph content (optionally with one or more other form of lercanidipine, such as, for example, lercanidipine hydrochloride crystalline Form (I), lercanidipine hydrochloride crystalline Form (II) or amorphous form) formulated according to, for example and without limitation, the compositions and dosage forms described herein.

The invention also contemplates a method of treating and preventing atherosclerotic lesions in arteries of a subject, the method comprising administering a therapeutically effective amount of isolated lercanidipine hydrochloride crystalline Form (III), isolated lercanidipine hydrochloride crystalline Form (IV), or combinations thereof to a subject in need of such treatment (optionally with other form of lercanidipine, such as, for example, lercanidipine hydrochloride crystalline Form (I), lercanidipine hydrochloride crystalline Form (II) or amorphous form).

### Solvates

The present invention contemplates novel solvates of lercanidipine hydrochoride. The solvates of the present invention include, but are not limited to, lercanidipine in combination with methylene chloride, methyl ethyl ketone, acetone, anisole, ethyl acetate, tetrahydrofuran, terbutyl methyl ether, isopropanol, 2-butanol, or heptane. These solvates are advantageous because they can be obtained under defined conditions.

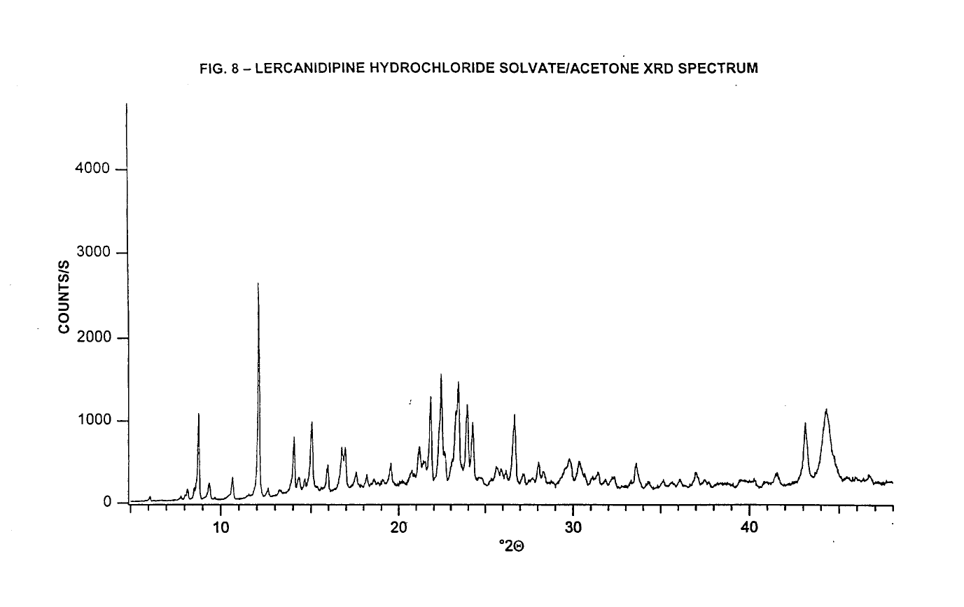

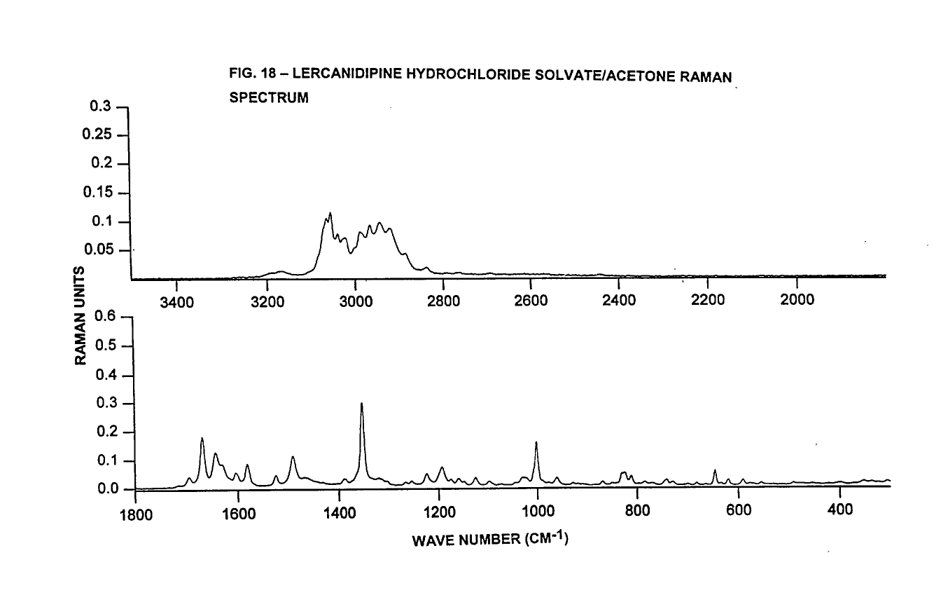

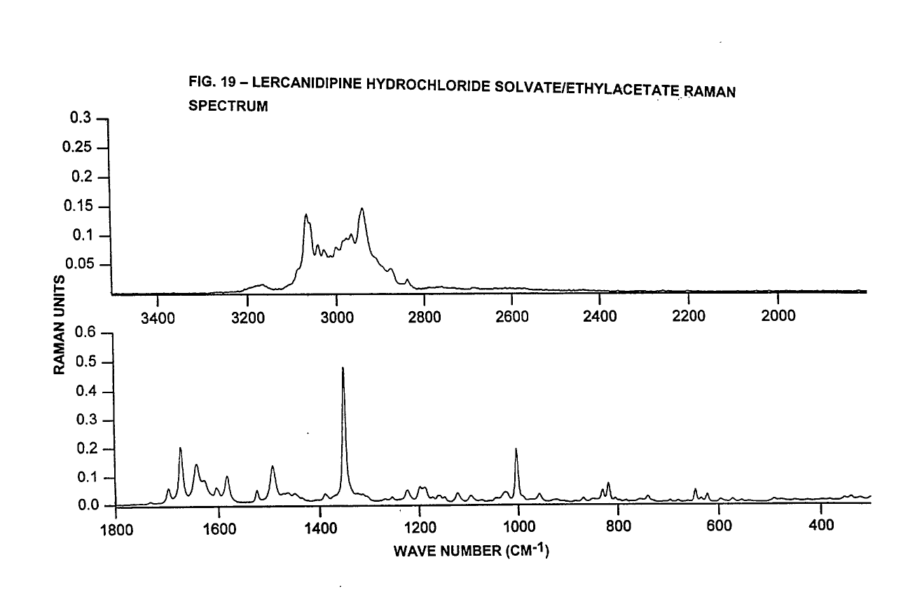

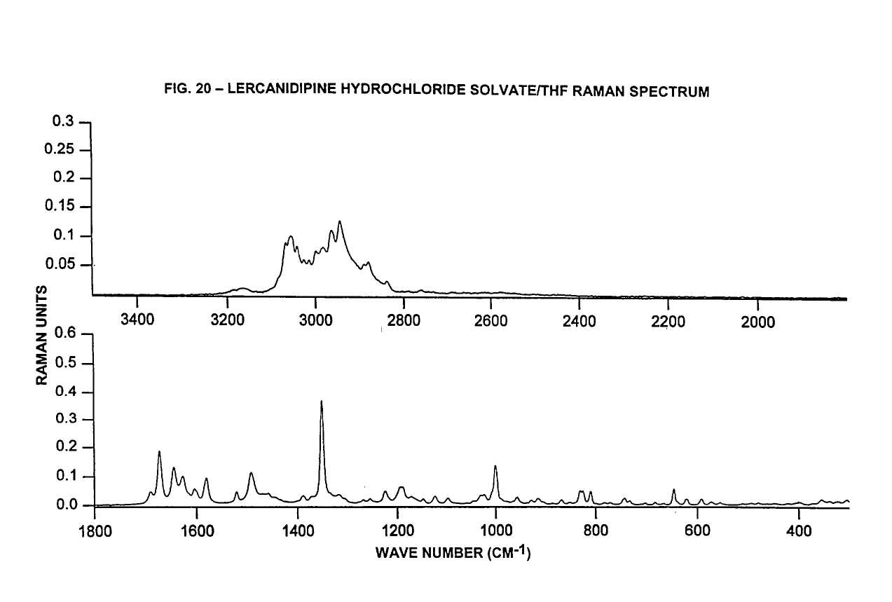

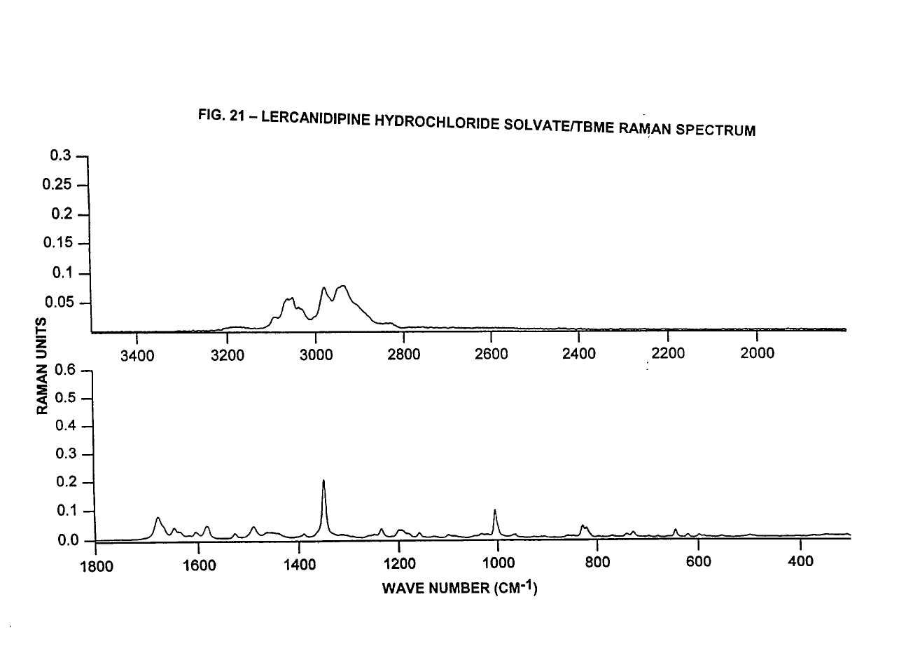

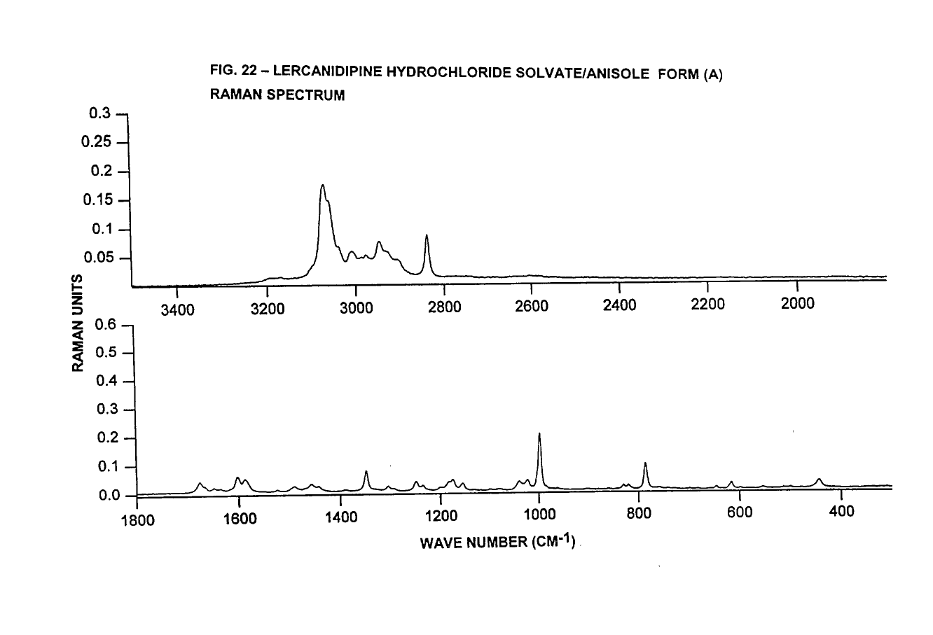

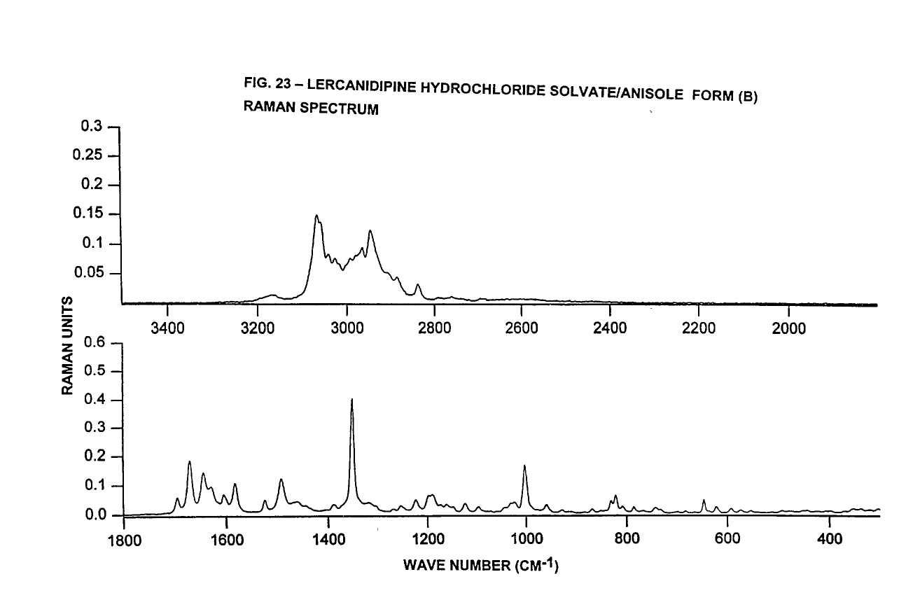

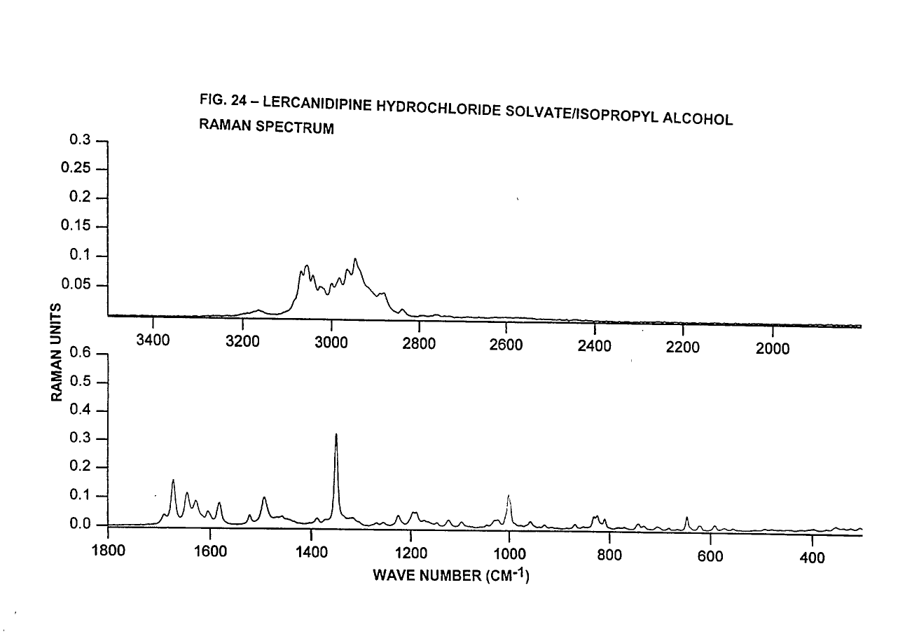

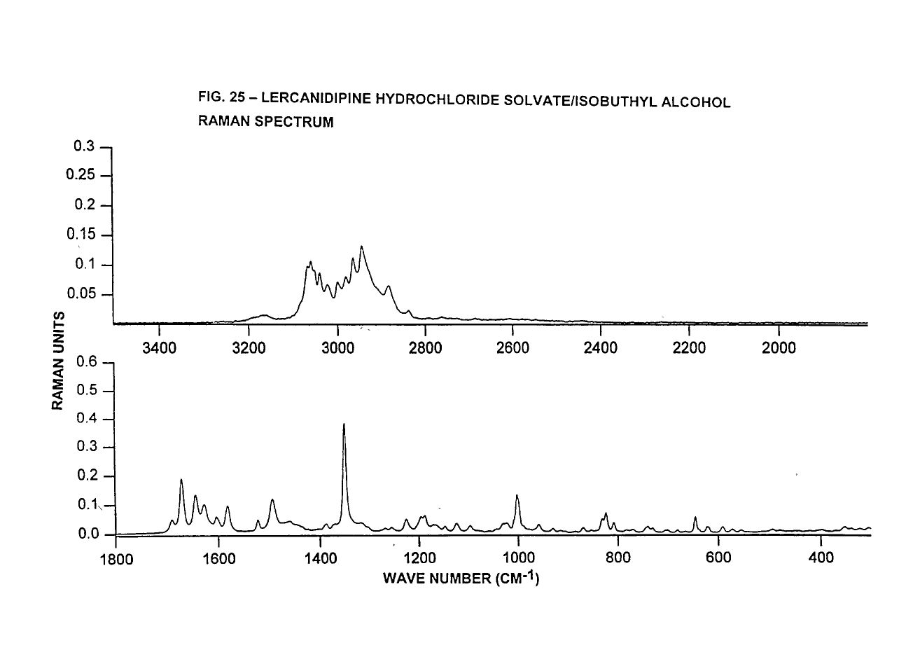

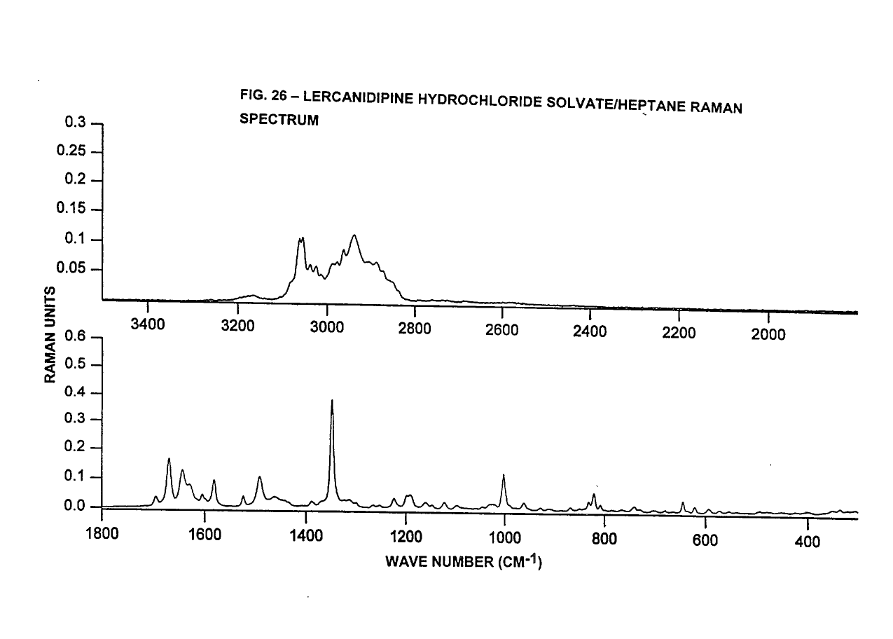

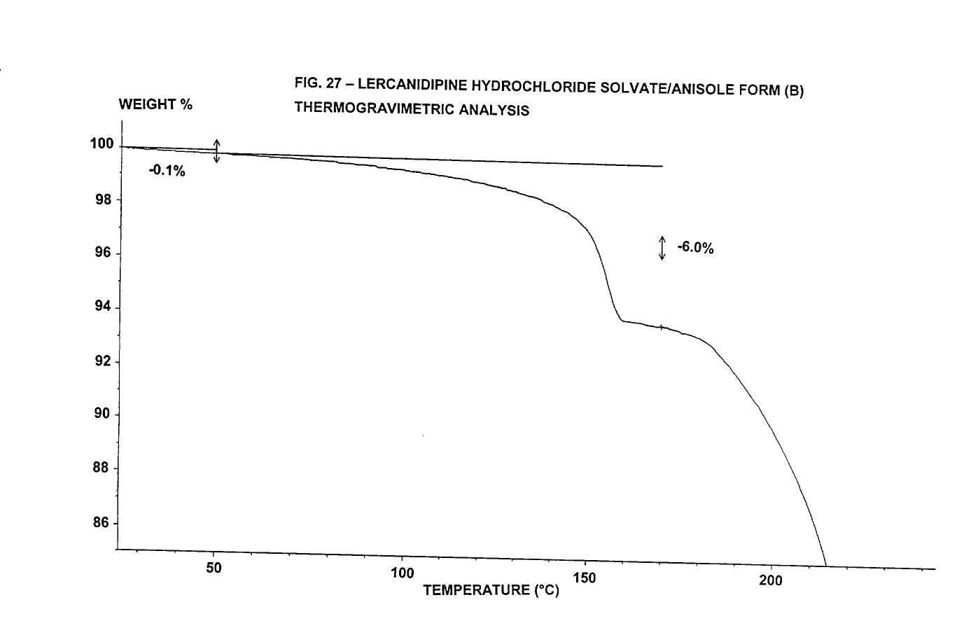

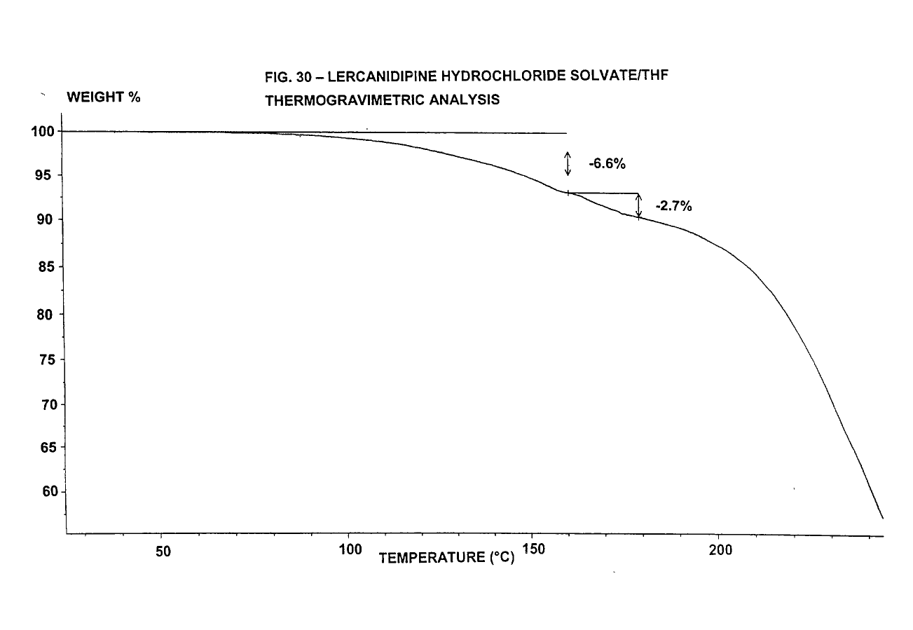

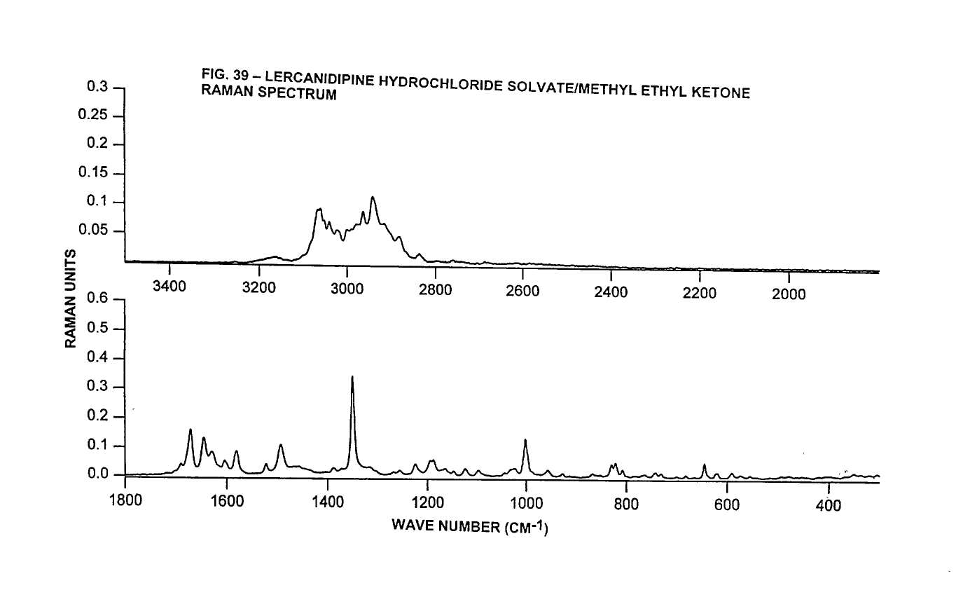

The present application contemplates any method that produces the solvates of the present invention. These solvates are defined by specific X-ray diffraction patterns (see FIGS. 8-16 and 38), Raman spectra (see FIGS. 18-26 and 39), and thermogravimetric results (see FIGS. 27-35 and 37). Specific methods of producing the solvates of the invention are disclosed herein.

A lercanidipine hydrochloride-methylene chloride solvate can be prepared with a method comprising the steps of:

(i) suspending crystalline Form (I) of lercanidipine hydrochloride that is described in Italian patent application no. MI 2001A 001726, filed Aug. 6, 2001, U.S. provisional application No. 60/367,789, filed Mar. 26, 2002, and co-pending U.S. patent application Ser. No. ______ of Bonifacio et al., for “NOVEL CRUDE AND CRYSTALLINE FORMS OF LERCANIDIPINE HYDROCHLORIDE,” (Docket no.: 4266/1J959-US 1), filed Aug. 6, 2002, in methylene chloride to produce a mixture;

(ii) placing the mixture of step (i) in a closed vessel and stirring under mild conditions, e.g., at a temperature between about 20 to 50° C. to produce a solid; and

(iii) isolating the solid produced in step (ii), e.g., by filtration.

In step (ii), stirring is typically performed for eight days. Similar results may be obtained, however, with longer or shorter times. The methods can also be practiced using lercanidipine crude form (C) as starting materia.

A lercanidipine hydrochloride-methyl ethyl ketone solvate can be prepared by:

(i′) dissolving lercanidipine hydrochloride crystalline Form (I) in methyl ethyl ketone to produce a solution;

(ii′) cooling the solution of step (i′) to preferably 20-25° C. while stirring and keeping the solution at the temperature for preferably at least two days to produce a solid; and

(iii′) filtering the solid and drying.

Step (i′) is preferably performed, for example, at 80° C. Also preferred is where the lercanidipine hydrochloride-methyl ethyl ketone solution of step (i′) comprises 0 to 5% (v/v) water.

Independent preferences for step (ii′) are cooling the solution to room temperature and stirring for at least two days. Further preferred are simultaneous preferences where the solution in step (ii′) is cooled to room temperature and stirred at room temperature for at least two days.

The preferred conditions for drying in step (iii′) are in an oven at 60° C. for 24 hours under vacuum.

The other solvates of the present invention can be obtained by suspending a lercanidipine hydrochloride-methylene chloride solvate prepared, for example and without limitation by the method in steps (i)-(iii), with a solvent selected from the group consisting of acetone, anisole, ethyl acetate, tetrahydrofuran, terbutyl methyl ether, isopropanol, 2-butanol, and heptane, at a temperature between 20 and 50° C. for 114 to 420 hours to produce a solid. The solid produced by this method is a novel solvate comprising lercanidipine hydrochloride with the solvent used in the reaction. Therefore, if heptane is used as the solvent then the final solvate would be lercanidipine hydrochloride-heptane.

It has been determined that when anisole is used as the solvate, that two different forms of the solvate may be produced ((a) and (b) forms). The differences between these forms are clear from their x-ray spectra. When using the method disclosed above lercanidipine hydrochloride-anisole (b) form is produced.

As an alternative method, the solvates can be prepared by suspending lercanidipine hydrochloride crystalline Form (III), which is described in further detail below, in a solvent selected from the group consisting of anisole, ethyl acetate, tetrahydrofuran, terbutyl methyl ether, or acetone to produce a solution. The solution that is prepared is kept under mild stirring in a closed vessel at a temperature between 20 and 50° C. for 114 to 420 hours to produce a solid. The solid that is formed is then filtered. When anisole is used as the solvent with this method, lercanidipine hydrochloride-anisole (a) form solvate is produced. In a preferred embodiment, the temperature is 20-50° C. and the solution is stirred from 114 to 420 hours.

In another alternative embodiment, the solvates of the present invention can be prepared by suspending crude lercanidipine hydrochloride Form (A) or (B) that are described in Italian patent application no. MI 2001A 001726, filed Aug. 6, 2001, U.S. provisional application No. 60/367,789, filed Mar. 26, 2002 and co-pending U.S. patent application Ser. No. ______ of Bonifacio et al., for “NOVEL CRUDE AND CRYSTALLINE FORMS OF LERCANIDIPINE HYDROCHLORIDE,” (Docket no.: 4266/1J959-US1), filed Aug. 6, 2002, in a solvent selected from the group consisting of anisole, ethyl acetate, tetrahydrofuran, terbutyl methyl ether, acetone, or methylene chloride to produce a suspension. The suspension is maintained under mild stirring in a closed vessel at a temperature between 20 and 50° C. for 114 to 420 hours. The solid that is produced is then filtered to give the final product. When anisole is used as the solvent in this method, lercandipine hydrochloride-anisole (a) form solvate is produced. In a preferred embodiment, the temperature is 20-50° C. and the solution is stirred for 114 to 420 hours.

The preparation of the these solvates, both starting from the solvate of lercanidipine hydrochloride or from the crude (A) or (B) or (C) forms, may be preferably carried out at room temperature. Alternatively, the method may include a series of thermal cycles performed after the solvent is added to lercanidipine. The length and number of the cooling and heating steps, as well as the temperatures, may be determined by one of ordinary skill in the art. In a preferred embodiment, the steps are about 3 hours each. In another embodiment, the heating step is performed at 35° C. and the cooling step is performed at 25° C. In a most preferred embodiment, the thermal cycle is composed of a cooling step at 25° C., heating step at 35° C., and a cooling step at 25° C. (written as 25° C.-35° C.-25° C.), where each step is about 3 hours. The number of cycles can preferably range from 10 to 20. Preferably, after completion of the final cycle the sample is stirred at a temperature of 25° C. for a period of time of 24-240 h.

### Crystalline Forms

Under specific conditions, removal of the solvents from the solvates disclosed above produces novel crystalline forms. These forms have been termed lercanidipine hydrochloride crystalline Form (III) and (IV).

The present application contemplates any and all methods that may be used to prepare the forms described herein. In the present application, preferred methods of preparing these forms are described.

In one method, evaporation of solvent from a solvate, under a nitrogen stream or under vacuum, produces lerecanidipine hydrochloride crystalline Form (III). A preferred set of conditions for evaporation is, without limitation, a vacuum of 1-0.01 mbarr for 20-30 hours at a temperature of 50-90° C. Preferably, the solvent is selected from the group consisting of methylene chloride, tetrahydrofuran, heptane, anisole, ethyl acetate, isopropanol and 2-butanol. In one embodiment, the solvate is selected from any of the solvates described herein, except lercanidipine hydrochloride-anisole (a) form that was described previously.

To prepare lercanidipine hydrochloride crystalline form (IV), acetone is removed from a lercanidipine hydrochloride-acetone solvate, under a nitrogen stream or under vacuum. A preferred set of conditions for acetone removal is, without limitation, a vacuum of 1-0.01 mbar for 20-30 hours at a temperature of 50-90° C.

### Pharmaceutical Compositions

The compounds and polymorphs of the present invention may be formulated into a pharmaceutical composition. The pharmaceutical composition may also include optional additives, such as a pharmaceutically acceptable carrier or diluent, a flavorant, a sweetener, a preservative, a dye, a binder, a suspending agent, a dispersing agent, a colorant, a disintegrant, an excipient, a film forming agent, a lubricant, a plasticizer, an edible oil or any combination of two or more of the foregoing.

Crystalline forms, e.g., lercanidipine hydrochloride crystalline Forms (III) or (IV), can undergo micronization, using any method known in the art. The average size of particle produced by this method are preferably D(50%)2-8 μm, D(90%)<15 μm.

Suitable pharmaceutically acceptable carriers or diluents include, but are not limited to, ethanol; water; glycerol; propylene glycol, aloe vera gel; allantoin; glycerin; vitamin A and E oils; mineral oil; PPG2 myristyl propionate; magnesium carbonate; potassium phosphate; vegetable oil; animal oil; and solketal.

Suitable binders include, but are not limited to, starch; gelatin; natural sugars, such as glucose, sucrose and lactose; corn sweeteners; natural and synthetic gums, such as acacia, tragacanth, vegetable gum, and sodium alginate; carboxymethylcellulose; hydroxypropylmethylcellulose; polyethylene glycol; povidone; waxes; and the like.

Suitable disintegrants include, but are not limited to, starch, e.g., corn starch, methyl cellulose, agar, bentonite, xanthan gum, sodium starch glycolate, crosspovidone and the like.

Suitable lubricants include, but are not limited to, sodium oleate, sodium stearate, sodium stearyl fumarate, magnesium stearate, sodium benzoate, sodium acetate, sodium chloride and the like.

A suitable suspending agent is, but is not limited to, bentonite, ethoxylated isostearyl alcohols, polyoxyethylene sorbitol and sorbitan esters, microcrystalline cellulose, aluminum metahydroxide, agar-agar and tragacanth, or mixtures of two or more of these substances, and the like.

Suitable dispersing and suspending agents include, but are not limited to, synthetic and natural gums, such as vegetable gum, tragacanth, acacia, alginate, dextran, sodium carboxymethylcellulose, methylcellulose, polyvinyl-pyrrolidone and gelatin.

Suitable film forming agents include, but are not limited to, hydroxypropylmethylcellulose, ethylcellulose and polymethacrylates.

Suitable plasticizers include, but are not limited to, polyethylene glycols of different molecular weights (e.g., 200-8000 Da) and propylene glycol.

Suitable colorants include, but are not limited to, ferric oxide(s), titanium dioxide and natural and synthetic lakes.

Suitable edible oils include, but are not limited to, cottonseed oil, sesame oil, coconut oil and peanut oil.

Examples of additional additives include, but are not limited to, sorbitol, talc, stearic acid, dicalcium phosphate and polydextrose.

### Unit Dosage Forms

The pharmaceutical composition may be formulated as unit dosage forms, such as tablets, pills, capsules, caplets, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. Unit dosage forms may be used for oral, parenteral, intranasal, sublingual or rectal administration, or for administration by inhalation or insufflation, transdermal patches, and a lyophilized composition. In general, any delivery of active ingredients that results in systemic availability of them can be used. Preferably the unit dosage form is an oral dosage form, most preferably a solid oral dosage form, therefore the preferred dosage forms are tablets, pills, caplets and capsules. Parenteral preparations also are preferred.

Solid unit dosage forms may be prepared by mixing an active agent of the present invention with a pharmaceutically acceptable carrier and any other desired additives as described above. The mixture is typically mixed until a homogeneous mixture of the active agents of the present invention and the carrier and any other desired additives is formed, i.e., until the active agent is dispersed evenly throughout the composition. In this case, the compositions can be formed as dry or moist granules.

Tablets or pills can be coated or otherwise compounded to form a unit dosage form which has delayed and/or prolonged action, such as time release and sustained release unit dosage forms. For example, the tablet or pill can comprise an inner dosage and an outer dosage component, the latter being in the form of a layer or envelope over the former. The two components can be separated by an enteric layer which serves to resist disintegration in the stomach and permits the inner component to pass intact into the duodenum or to be delayed in release.

Biodegradable polymers for controlling the release of the active agents, include, but are not limited to, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

For liquid dosage forms, the active substances or their physiologically acceptable salts are brought into solution, suspension or emulsion, optionally with the usually employed substances such as solubilizers, emulsifiers or other auxiliaries. Solvents for the active combinations and the corresponding physiologically acceptable salts, can include water, physiological salt solutions or alcohols, e.g., ethanol, propane-diol or glycerol. Additionally, sugar solutions such as glucose or mannitol solutions may be used. A mixture of the various solvents mentioned may further be used in the present invention.

A transdermal dosage form also is contemplated by the present invention. Transdermal forms may be a diffusion-driven transdermal system (transdermal patch) using either a fluid reservoir or a drug-in-adhesive matrix system. Other transdermal dosage forms include, but are not limited to, topical gels, lotions, ointments, transmucosal systems and devices, and iontohoretic (electrical diffusion) delivery system. Transdermal dosage forms may be used for timed release and sustained release of the active agents of the present invention.

Pharmaceutical compositions and unit dosage forms of the present invention for administration parenterally, and in particular by injection, typically include a pharmaceutically acceptable carrier, as described above. A preferred liquid carrier is vegetable oil. Injection may be, for example, intravenous, intrathecal, intramuscular, intraruminal, intratracheal, or subcutaneous.

The active agent also can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The polymorphs of the present invention also may be coupled with soluble polymers as targetable drug carriers. Such polymers include, but are not limited to, polyvinyl-pyrrolidone, pyran copolymer, polyhydroxypropylmethacryl-amidephenol, polyhydroxy-ethylaspartamidephenol, and polyethyl-eneoxideopolylysine substituted with palmitoyl residues.

The present application further discloses pharmaceutical formulations and unit dosage forms that comprise one of the isolated polymorphs of the present invention or a mixture thereof of predetermined polymorph content. Dosage forms with predetermined amounts of lercanidipine hydrochloride may be formulated starting with compositions with known quantities of lercanidipine hydrochloride using methods well known in the art. In a preferred embodiment a dosage form is obtained by mixing compositions comprising known quantities of crystalline lercanidipine hydrochloride, e.g., Form (III) or (IV), optionally including other forms of crystalline lercanidipine hydrochloride, e.g., Form (I) or (II), or non-crystalline forms or lercanidipine hydrochloride, e.g., amorphous. Further preferred is where a dosage form with predetermined amounts of crystalline lercanidipine hydrochloride is formulated by mixing compositions comprising essentially pure crystalline lercanidipine hydrochloride are mixed to form dosage forms comprising a predetermined ratio of crystalline Forms (III) and (IV).

Dosage forms preferably comprise a predetermined amount of any one of crystalline lercanidipine hydrochloride Form (I), (II), (III) or (IV). Also preferred are dosage forms that simultaneously comprise predetermined amounts of at least two crystalline lercanidipine hydrochloride Forms, e.g., Forms (I) and (II), Forms (I) and (III), Forms (I) and (IV), Forms (II) and (III), Forms (II) and (IV), or Forms (III) and (IV). Also preferred are dosage forms that simultaneously comprise predetermined amounts of at least three crystalline lercanidipine hydrochloride Forms, e.g., Forms (I), (II) and (III), Forms (I), (II) and (IV), Forms (I), (III) and (IV), or Forms (II), (III) and (IV). Also preferred are dosage forms that simultaneously comprise predetermined amounts of at least four crystalline lercanidipine hydrochloride Forms, e.g., Forms (I), (II), (III) and (IV). Each of the aforementioned may optionally include other forms of lercanidipine such as, for example and without limitation, indeterminate or amounts of crystalline lercanidipine hydrochloride, e.g., Forms (I), (II), (III) and (IV), that have not been predetermined, or other forms of lercanidipine, e.g., crude or amorphous.

### Administration

The pharmaceutical composition or unit dosage forms of the present invention may be administered by a variety of routes such as intravenous, intratracheal, subcutaneous, oral, mucosal parenteral, buccal, sublingual, ophthalmic, pulmonary, transmucosal, transdermal, and intramuscular. Unit dosage forms also can be administered in intranasal form via topical use of suitable intranasal vehicles, or via transdermal routes, using of transdermal skin patches known to those of ordinary skill in the art. Oral administration is preferred.

The pharmaceutical composition or unit dosage forms of the present invention may be administered to an animal, preferably a human being, in need of antihypertensive treatment. The pharmaceutical composition or unit dosage form of the present invention may be administered according to a dosage and administration regimen defined by routine testing in light of the guidelines given above in order to obtain optimal antihypertensive activity and a decreased in blood pressure while minimizing toxicity or side-effects for a particular patient. However, such fine turning of the therapeutic regimen is routine in light of the guidelines given herein.

The dosage of the composition containing solvates, polymorphs or mixtures thereof, of the present invention may vary according to a variety of factors such as underlying disease state, the individual's condition, weight, sex and age and the mode of administration. For oral administration, the pharmaceutical compositions can be provided in the form of scored or unscored solid unit dosage forms.

A pharmaceutical composition comprising (1) lercanidipine hydrochloride, where the lercanidipine hydrochloride is selected from the group consisting of isolated lercanidipine hydrochloride crystalline Form (III), isolated lercanidipine hydrochloride crystalline Form (IV), or combinations thereof of predetermined polymorph composition; and (2) at least one component selected from the group consisting of a pharmaceutically acceptable carrier or diluent, a flavorant, a sweetener, a preservative, a dye, a binder, a suspending agent, a dispersing agent, a colorant, a disintegrant, an excipient, a diluent, a lubricant, a plasticizer, and an edible oil. In a preferred embodiment, the pharmaceutical composition or dosage form comprises 0.1 to 400 mg lercanidipine hydrochloride for all uses disclosed herein. Preferably, the composition or dosage form comprises 1 to 200 mg lercanidipine hydrochloride. More preferably, the composition or dosage form comprises 5 to 40 mg lercanidipine hydrochloride. Smaller amounts may be selected when a preferred enantiomer having higher activity for a particular therapeutic goal is used.

The pharmaceutical composition or unit dosage form may be administered in a single daily dose, or the total daily dosage may be administered in divided doses. In addition, co-administration or sequential administration of other active agents may be desirable. The solvates, polymorphs and mixtures thereof of the invention may be combined with any known drug therapy, preferably for treatment of hypertension. For example, bimodal therapy involving in addition a diuretic, a β-receptor blocker, an ACE inhibitor or an angiotensin II receptor antagonist is contemplated by the present invention (see, e.g., U.S. Provisional Application No. 60/344,601, filed Oct. 23, 2001 and Italian Application No. MI 2001 A 002136 filed Oct. 16, 2001).

For combination therapy the compounds may initially be provided as separate dosage forms until an optimum dosage combination and administration regimen is achieved. Therefore, the patient may be titrated to the appropriate dosages for his/her particular hypertensive condition. After the appropriate dosage of each of the compounds is determined to achieve a decrease of the blood pressure without untoward side effects, the patient then may be switched to a single dosage form containing the appropriate dosages of each of the active agents, or may continue with a dual dosage form.

The exact dosage and administration regimen utilizing the combination therapy of the present invention is selected in accordance with a variety of factors including type, species, age, weight, sex and medical condition of the patient; the severity and etiology of the hypertension to be treated; the route of administration; the renal and hepatic function of the patient; the treatment history of the patient; and the responsiveness of the patient. Optimal precision in achieving concentrations of compounds within the range that yields efficacy without toxicity requires a regimen based on the kinetics of the drug's availability to target sites. This involves a consideration of the absorption, distribution, metabolism, excretion of a drug, and responsiveness of the patient to the dosage regimen. However, such fine tuning of the therapeutic regimen is routine in light of the guidelines given herein.

A pharmaceutical composition for parenteral administration contains not below 0.1%, preferably from about 0.5% to about 30%, by weight of a solvate, polymorph or mixture of the present invention, based upon the total weight of the pharmaceutical composition. Individual isolated polymorphs are preferred for parenteral administration.

Generally, transdermal dosage forms contain from about 0.01% to about 100% by weight of the active agents, based upon 100% total weight of the dosage.

In a preferred embodiment of the present invention, the composition is administered daily to the patient. In a further preferred embodiment, the pharmaceutical composition or dosage form 0.1 to 400 mg lercanidipine hydrochloride. Preferably, the composition or dosage form comprises 1 to 200 mg lercanidipine hydrochloride. More preferably, the composition or dosage form comprises 5 to 40 mg lercanidipine hydrochloride.

### EXAMPLES

### Example 1

Preparation of the Solvate of Lercanidipine Hydrochloride with Methylene Chloride

Lercanidipine hydrochloride Form (I) (5.34 g), prepared as described in Italian patent application no. MI 2001A 001726, filed Aug. 6, 2001, U.S. provisional application No. 60/367,789, filed Mar. 26, 2002 and co-pending U.S. patent application Ser. No. ______ of Bonifacio et al., for “NOVEL CRUDE AND CRYSTALLINE FORMS OF LERCANIDIPINE HYDROCHLORIDE” (Docket no.:4266/1J959-US1), filed Aug. 6, 2002, was combined with 20 ml of methylene chloride in a closed vessel, the suspension was kept under mild stirring for 192 hours at 20-25° C. to produce a solid. The solid was then filtered with a glass filter G4 and washed with fresh methylene chloride. A product of 7.4 g of lercanidipine hydrochloride-methylene chloride solvate (1:1 mol/mol) was obtained.

### Example 2

Preparation Lercanidipine Hydrochloride Form (III)

3.9 g of solvate of lercanidipine hydrochloride with methylene chloride solvate, prepared by the method of Example 1, was placed in a glove hood under constant nitrogen stream (25 l/h) at ambient temperature. The sample was then dried at 90° C. and 1 millibar, then placed in a stoppered flask and isolated with parafilm.

Examples 3-27 Describe Preparation of Solvates of Lercanidipine Hydrochloride with Solvents other than Methylene Chloride

### Examples 3-6

Preparation of Solvates of Lercanidipine Hydrochloride with Anisole, Ethyl Acetate and Terbutyl Methyl Ether

The solvate of lercanidipine hydrochloride with methylene chloride prepared as described in Example 1, the lercanidipine hydrochloride crystalline form (III) obtained as described in Example 2, or the lercanidipine crude (A) or (B) forms prepared as described in Italian patent application no. MI 2001A 001726, filed Aug. 6, 2001 and U.S. provisional application No. 60/367,789, filed Mar. 26, 2002, were introduced in a closed vessel together with a solvent chosen from the group consisting of anisole, ethyl acetate and terbutyl methyl ether, under mild stirring, with 10-20 thermal cycles:25° C.-35° C.-25° C. (3 hours each). After these thermal cycles the samples were kept at 25° C. for 24-240 hours. The solvate was then isolated by filtration. The solvent used, the concentration of the starting product in the solvation solvent, and the solvate stoichiometry are shown in Table 1.

### Examples 7-9

Preparation of Solvates of Lercanidipine Hydrochloride with Isopropanol, 2-butanol, Heptane

The solvate of lercanidipine hydrochloride with methylene chloride prepared as described in Example 1 was placed in a closed vessel together with a solvent chosen from the group consisting of isopropanol, 2-butanol and heptane, kept under mild stirring and subjected to 10-20 thermal cycles 25° C.-35° C.-25° C. (heating step 3 hours, cooling step 3 hours). After these thermal cycles the sample was kept at 25° C. for 24-240 hours and then filtered. The solvent used, the concentrations of the starting product in the solvation solvent and the solvate stoichiometry are shown in Table 1.

### Examples 10-11

Preparation of Solvates of Lercanidipine Hydrochloride with Acetone and Tetrahydrofuran

The solvate of lercanidipine hydrochloride with methylene chloride, prepared as described in Example 1, and a solvent chosen from the group consisting of acetone and tetrahydrofuran were placed in a closed vessel, kept under mild stirring and subjected to 10-20 thermal cycles:25° C.-35° C.-25° C. (heating step 3 hours, cooling step 3 hours). After these thermal cycles the samples were kept at 25° C. for 24-240 hours and then filtered. The solvent used, the concentrations of the starting product in the solvation solvent and the solvate stoichiometry are shown in Table 1.

TABLE 1

| | | | SOLID SOLVATE |
|---|---|---|---|
| | | | OBTAINED |
| | CONCENTRATION | | [content of solvent:lercanidipine |
| EXAMPLE | (mg/ml) | SOLVENT | hydrochloride (mole/mole)] |
| 3 | 500 | Anisole | lercanidipine hydrochloride- |
| | | | anisole (b) form |
| | | | [0.4] |
| 4 | 508 | Ethyl acetate | lercanidipine hydrochloride- |
| | | | ethyl acetate |
| | | | [1] |
| 5 | 164 | Anisole | lercanidipine hydrochloride- |
| | | | anisole (a) form |
| | | | [0.4] |
| 6 | 229 | Terbutyl methyl | lercanidipine hydrochloride- |
| | | ether | terbutyl methyl ether |
| | | | [0.8] |
| 7 | 447 | Isopropanol | lercanidipine hydrochloride- |
| | | | isopropanol |
| | | | [1] |
| 8 | 390 | 2-butanol | lercanidipine hydrochloride-2- |
| | | | butanol |
| | | | [0.8] |
| 9 | 348 | Heptane | lercanidipine hydrochloride- |
| | | | heptane |
| | | | [0.9] |
| 10 | 297 | Acetone | lercanidipine hydrochloride- |
| | | | acetone |
| | | | [1.2] |
| 11 | 308 | Tetrahydrofuran | lercanidipine hydrochloride- |
| | | | tetrahydrofuran |
| | | | [0.9] |

### Examples 12-19

Preparation of Solvates of Lercanidipine Hydrochloride with 2-propanol, 2-butanol, Tetrahydrofuran, Terbutyl Methyl Ether, Anisole, Acetone, Ethyl Acetate, Heptane, using Lercanidipine-Hydrochloride-Methylene Chloride

The solvate of lercanidipine hydrochloride with methylene chloride, obtained as described Example 1, was suspended in a closed vessel in a solvent chosen from the group consisting of 2-propanol, 2-butanol, tetrahydrofuran, terbutyl methyl ether, anisole, acetone, ethyl acetate and heptane. The suspension that was produced was stirred at 20-50° C. for 114-420 hours to produce the solvates. The solvates were then filtered. The solvent used, the concentration of the starting product in the solvation solvent, and the solvate obtained are shown in Table 2.

TABLE 2

| | CONCENTRATION | | SOLID SOLVATE |
|---|---|---|---|
| EXAMPLE | (mg/ml) | SOLVENT | OBTAINED |
| 12 | 320 | 2-propanol | lercanidipine |
| | | | hydrochloride- |
| | | | 2-propanol |
| 13 | 323 | 2-butanol | lercanidipine |
| | | | hydrochloride- |
| | | | 2-butanol |
| 14 | 323 | Tetrahydrofuran | lercanidipine |
| | | | hydrochloride- |
| | | | tetrahydrofuran |
| 15 | 306 | Terbutyl methyl | lercanidipine |
| | | ether | hydrochloride-terbutyl |
| | | | methyl ether |
| 16 | 306 | Anisole | lercanidipine |
| | | | hydrochloride-anisole (b) |
| | | | form |
| 17 | 320 | Acetone | lercanidipine |
| | | | hydrochloride-acetone |
| 18 | 320 | Ethyl acetate | lercanidipine |
| | | | hydrochloride- |
| | | | ethyl acetate |
| 19 | 330 | Heptane | lercanidipine |
| | | | hydrochloride-heptane |

### Examples 20-24

Preparation of Solvates of Lercanidipine Hydrochloride with the Following Solvents Chosen in the Group Comprising:Tetrahydrofuran, Terbutyl Methyl Ether, Anisole, Acetone, Ethyl Acetate

Lercanidipine hydrochloride crystalline form (III), obtained as described in Example 2, was suspended in a solvent chosen from the group consisting of tetrahydrofuran, terbutyl methyl ether, anisole, acetone and ethyl acetate. The suspension then was stirred at 20-50° C. for 114-240 hours. The solvent used, the concentration of the starting product in the solvent and the solvate obtained are shown in the following Table 3.

TABLE 3

| | CONCENTRATION | | |
|---|---|---|---|
| EXAMPLE | (mg/ml) | SOLVENT | SOLID OBTAINED |
| 20 | 317 | Tetrahydrofuran | lercanidipine hydrochloride- |
| | | | tetrahydrofuran |
| 21 | 313 | Terbutyl methyl | lercanidipine hydrochloride- |
| | | ether | terbutyl methyl ether |
| 22 | 317 | Anisole | lercanidipine hydrochloride- |
| | | | anisole (a) form |
| 23 | 313 | Acetone | lercanidipine hydrochloride- |
| | | | acetone |
| 24 | 327 | Ethyl acetate | lercanidipine hydrochloride- |
| | | ethyl acetate | |

### Examples 25-33

De-Solvation of the Solvates Obtained in Examples 3-11

The solvent was removed from the solvates by heating under vacuum. The starting solvate (also indicated with the number of the preparation example), the operating conditions applied in the removal of the inclusion solvent and the crystalline form of lercanidipine hydrochloride obtained are shown in Table 4.

TABLE 4

| | | | | CRYSTALLINE |
|---|---|---|---|---|
| | | STARTING | SOLVENT | LERCANIDIPINE |
| | | SOLVATE | REMOVAL | HCl FORM |
| EXAMPLE | STARTING SOLVATE | PREPARATION | CONDITIONS | OBTAINED* |
| 25 | lercanidipine | Example 3 | 90° C./<1 mbar/ | Form (III) |
| | hydrochloride-anisole | | 24 hours | |
| | (b form) | | | |
| 26 | lercanidipine | Example 4 | 90° C./<1 mbar/ | Form (III) |
| | hydrochloride-ethyl | | 24 hours | |
| | acetate | | | |
| 27 | lercanidipine | Example 5 | 50° C./<1 mbar/ | Form (I) |
| | hydrochloride-anisole | | 24 hours | |
| | (a form) | | | |
| 28 | lercanidipine | Example 6 | 90° C./<1 mbar/ | Form (I) |
| | hydrochloride-terbutyl | | 24 hours | |
| | methyl ether | | | |
| 29 | lercanidpine | Example 10 | 90° C./<1 mbar/ | Form (IV) |
| | hydrochloride-acetone | | 24 hours | |
| 30 | lercanidipine | Example 11 | 90° C./<1 mbar/ | Form (III) |
| | hydrochloride- | | 24 hours | |
| | tetrahydrofurane | | | |
| 31 | lercanidipine | Example 7 | 90° C./<1 mbar/ | Form (III) |
| | hydrochloride- | | 22 hours | |
| | isopropanol | | | |
| 32 | lercanidipine | Example 8 | 90° C./<1 mbar/ | Form (III) |
| | hydrochloride-2-butanol | | 22 hours | |
| 33 | lercanidipine | Example 9 | 90° C./<1 mbar/ | Form (III) |
| | hydrochloride-heptane | | 22 hours | |

*determined by Raman spectroscopy and X-ray diffraction

### Example 34

Preparation of the Solvate Lercanidipine Hydrochloride-Methyl Ethyl Ketone

100 g of lercanidipine hydrochloride crystalline Form (I) was suspended in 250 ml of methyl ketone/water (95/5) and heated at 80° C. until complete dissolution. The solution was cooled under stirring, kept at room temperature, and then filtered. The product was dried in an oven at 60° C. under vacuum (about 200 mmHg). 93 g of product was obtained having a lercanidipine hydrochloride-methyl ethyl ketone content of 1:0.7 (mole/mole).

### Example 35

X-Ray Diffraction

The new crystalline forms are hereinafter identified by their X-ray spectrums.

Philips PW 1710 and Philips X pert PW 3040 powder diffractometer (Copper Kα radiation) were used, under the following typical conditions:about 5-70 mg sample (without any previous treatment) with application of a slight pressure to obtain a flat surface. Ambient air atmosphere. 0.02° 2θ stepsize, 2 sec step-1, 2-50 2θ.

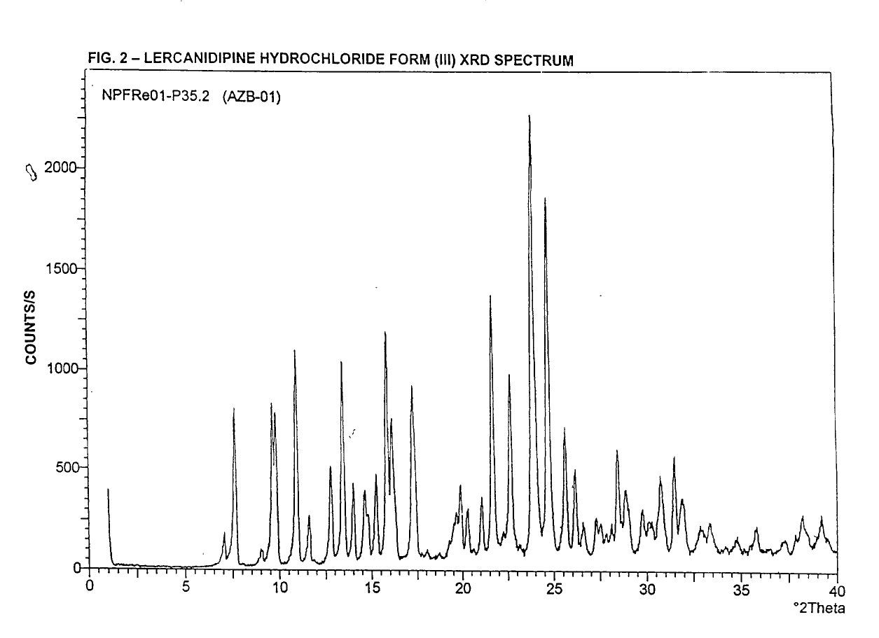

Lercanidipine hydrochloride crystalline form (III) showed an X-ray diffraction image at wavelength Kα as expressed in Table 5 and shown in FIG. 2.

TABLE 5

| d (Å) | Relative intensity (%) | Angle (°2 θ) |
|---|---|---|
| 11.5 | 39 | 7.7 |
| 9.1 | 38 | 9.7 |
| 9.0 | 37 | 9.8 |
| 8.0 | 50 | 11.0 |
| 6.6 | 48 | 13.5 |
| 5.58 | 57 | 15.9 |
| 5.49 | 34 | 16.1 |
| 5.13 | 43 | 17.3 |
| 4.09 | 63 | 21.7 |
| 3.92 | 43 | 22.7 |
| 3.72 | 100 | 23.9 |
| 3.60 | 85 | 24.7 |
| 3.47 | 31 | 25.6 |

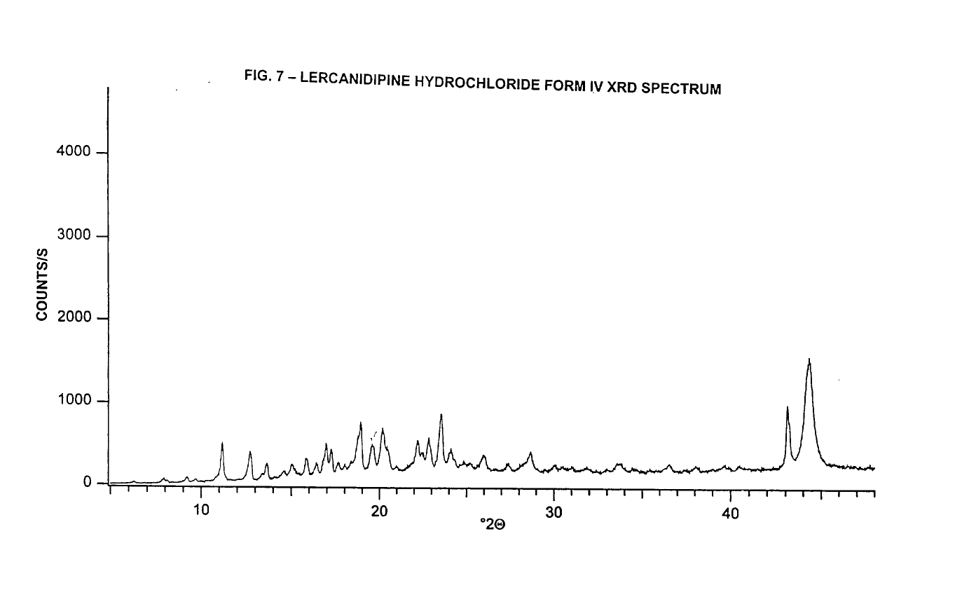

Lercanidipine hydrochloride crystalline form (IV) showed an X-ray diffraction image, at wavelength Kα as expressed in Table 6 and shown in FIG. 7.

TABLE 6

| d (Å) | Relative intensity (%) | Angle (°2 θ) |
|---|---|---|
| 7.9 | 71 | 11.2 |
| 6.9 | 53 | 12.7 |
| 5.21 | 57 | 17.0 |
| 5.13 | 46 | 17.3 |
| 4.73 | 66 | 18.8 |
| 4.69 | 95 | 18.9 |
| 4.53 | 53 | 19.6 |
| 4.40 | 81 | 20.2 |
| 4.34 | 43 | 20.4 |
| 3.99 | 44 | 22.2 |
| 3.89 | 52 | 22.8 |
| 3.77 | 100 | 23.6 |
| 3.69 | 35 | 24.1 |

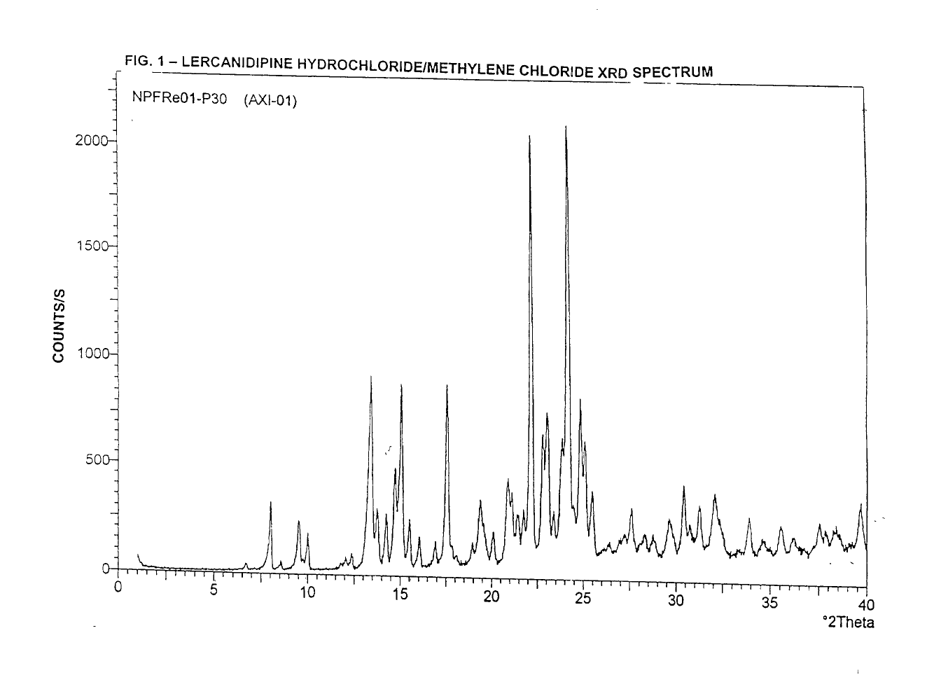

The solvate of lercanidipine hydrochloride with methylene chloride showed an X-ray diffraction image, at wavelength Kα as expressed in Table 7 and shown in FIG. 1.

TABLE 7

| d (Å) | Relative intensity (%) | Angle (°2 θ) |
|---|---|---|
| 6.6 | 40 | 13.4 |
| 5.87 | 42 | 15.1 |
| 5.04 | 39 | 17.6 |
| 4.00 | 96 | 22.2 |
| 3.90 | 29 | 22.8 |
| 3.86 | 34 | 23.0 |
| 3.67 | 100 | 24.2 |
| 2.04 | 31 | 44.4 |

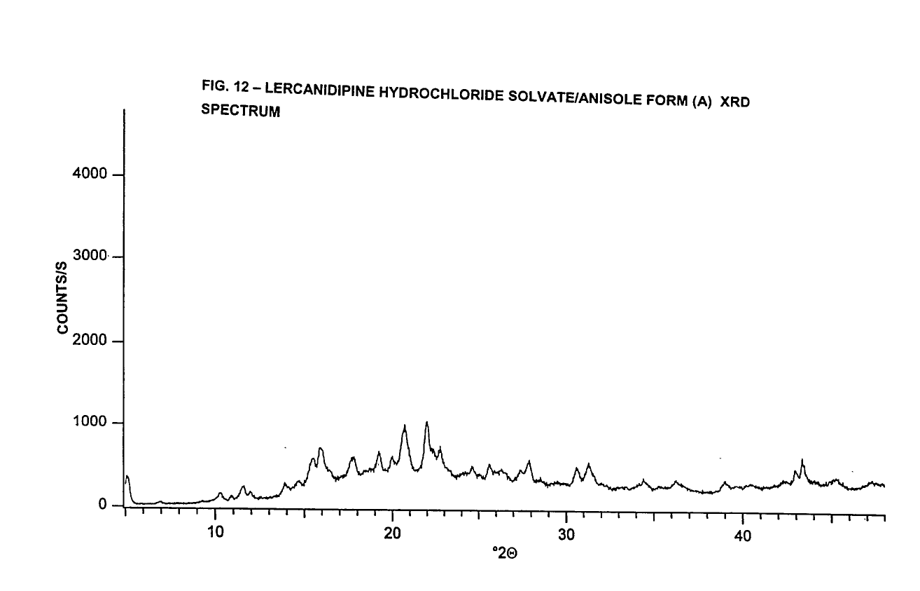

The solvate of lercanidipine hydrochloride with anisole (a) form showed an X-ray diffraction image, at wavelength Kα as expressed in Table 8 and shown in FIG. 12.

TABLE 8

| d (Å) | Relative intensity (%) | Angle (°2 θ) |
|---|---|---|
| 17.4 | 62 | 5.1 |
| 7.6 | 34 | 11.6 |
| 5.71 | 43 | 15.5 |
| 5.57 | 58 | 15.9 |
| 4.99 | 47 | 17.7 |
| 4.62 | 40 | 19.2 |
| 4.44 | 29 | 20.0 |
| 4.28 | 98 | 20.8 |
| 4.04 | 100 | 22.0 |
| 3.19 | 43 | 27.9 |
| 2.92 | 36 | 30.6 |
| 2.86 | 42 | 31.3 |

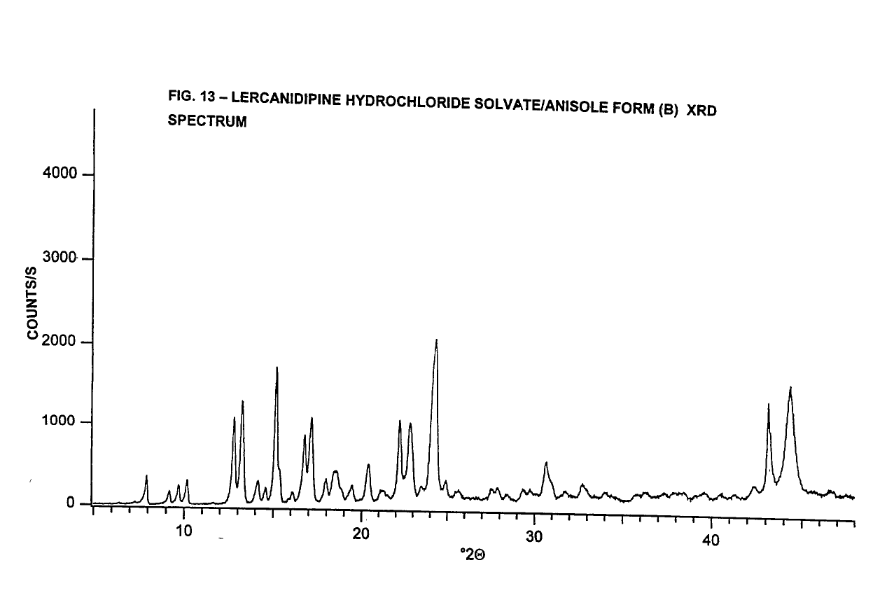

The solvate of lercanidipine hydrochloride with anisole (b) form showed an X-ray diffraction image, at wavelength Kα expressed in Table 9 and shown in FIG. 13.

TABLE 9

| d (Å) | Relative intensity (%) | Angle (°2 θ) |
|---|---|---|
| 6.9 | 49 | 12.8 |
| 6.7 | 63 | 13.3 |
| 5.82 | 86 | 15.2 |
| 5.27 | 41 | 16.8 |
| 5.15 | 53 | 17.2 |
| 4.00 | 47 | 22.2 |
| 3.89 | 46 | 22.8 |
| 3.66 | 100 | 24.3 |

The solvate of lercanidipine hydrochloride with acetone showed an X-ray diffraction image, at wavelength Kα as expressed in Table 10 and shown in FIG. 8.

TABLE 10

| d (Å) | Relative intensity (%) | Angle (°2 θ) |
|---|---|---|
| 10.1 | 42 | 8.8 |
| 7.3 | 100 | 1 2.1 |
| 5.87 | 31 | 15.1 |
| 4.07 | 41 | 21.8 |
| 3.96 | 52 | 22.4 |
| 3.79 | 49 | 23.5 |
| 3.71 | 37 | 24.0 |
| 3.34 | 33 | 26.7 |

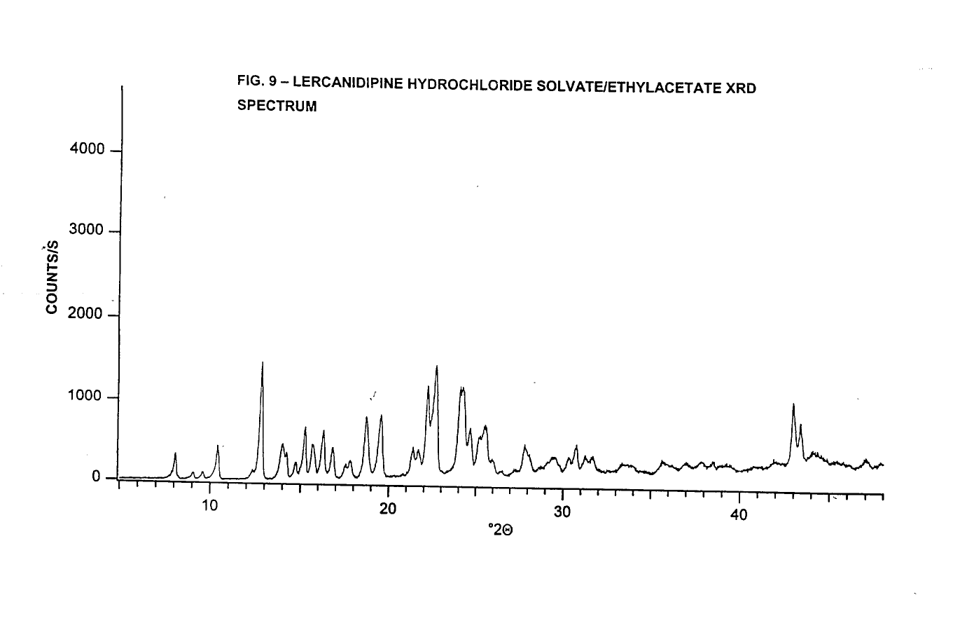

The solvate of lercanidipine hydrochloride with ethyl acetate showed an X-ray diffraction image, at wavelength Kα as expressed in Table 11 and shown in FIG. 9.

TABLE 11

| d (Å) | Relative intensity (%) | Angle (°2 θ) |
|---|---|---|
| 6.9 | 100 | 12.8 |
| 6.3 | 29 | 14.0 |
| 5.80 | 45 | 15.3 |
| 5.65 | 31 | 15.7 |
| 5.43 | 44 | 16.3 |
| 4.74 | 53 | 18.7 |
| 4.53 | 49 | 19.6 |
| 4.00 | 84 | 22.2 |
| 3.91 | 91 | 22.7 |
| 3.67 | 77 | 24.2 |
| 3.60 | 34 | 24.7 |
| 3.53 | 34 | 25.2 |
| 3.49 | 43 | 25.5 |

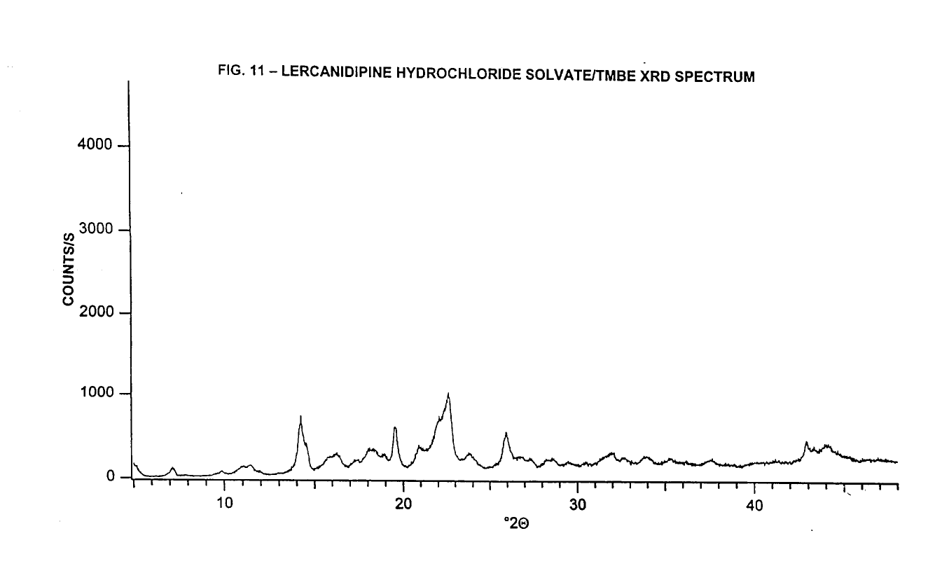

The solvate of lercanidipine hydrochloride with terbutyl methyl ether showed an X-ray diffraction image, at wavelength Kα, as expressed in Table 12 and shown in FIG. 11.

TABLE 12

| d (Å) | Relative intensity (%) | Angle (°2 θ) |
|---|---|---|
| 6.2 | 77 | 14.2 |
| 4.88 | 29 | 18.2 |
| 4.52 | 64 | 19.6 |
| 4.02 | 48 | 22.1 |
| 3.93 | 100 | 22.6 |
| 3.43 | 46 | 26.0 |

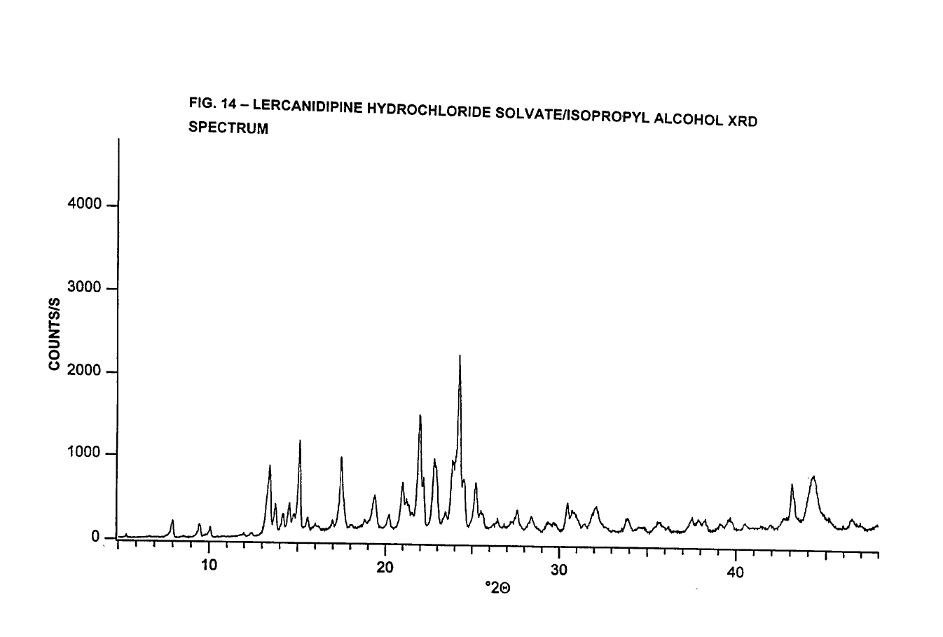

The solvate of lercanidipine hydrochloride with isopropanol showed an X-ray diffraction image, at wavelength Kα as expressed in Table 13 and shown in FIG. 14.

TABLE 13

| d (Å) | Relative intensity (%) | Angle (°2 θ) |
|---|---|---|
| 6.6 | 35 | 13.5 |
| 5.85 | 48 | 15.1 |
| 5.06 | 41 | 17.5 |
| 4.04 | 64 | 22.0 |
| 3.90 | 39 | 22.8 |
| 3.72 | 37 | 23.9 |
| 3.67 | 100 | 24.2 |

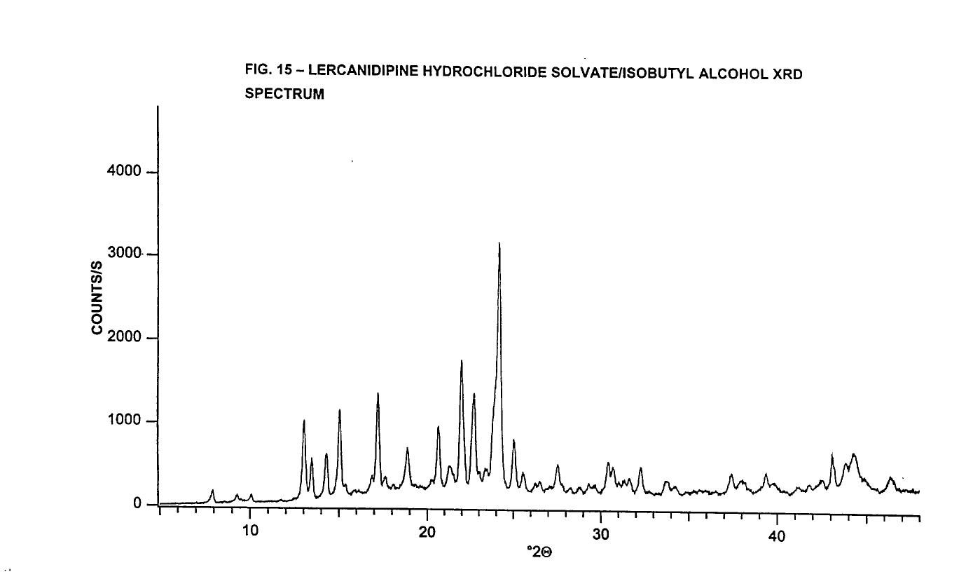

The solvate of lercanidipine hydrochloride with 2-butanol showed an X-ray diffraction image, at wavelength Kα the image as expressed in Table 14 and shown in FIG. 15.

TABLE 14

| d (Å) | Relative intensity (%) | Angle (°2 θ) |
|---|---|---|
| 6.8 | 34 | 13.1 |
| 5.86 | 36 | 15.1 |
| 5.13 | 42 | 17.3 |
| 4.03 | 51 | 22.0 |
| 3.90 | 36 | 22.8 |
| 3.67 | 100 | 24.2 |

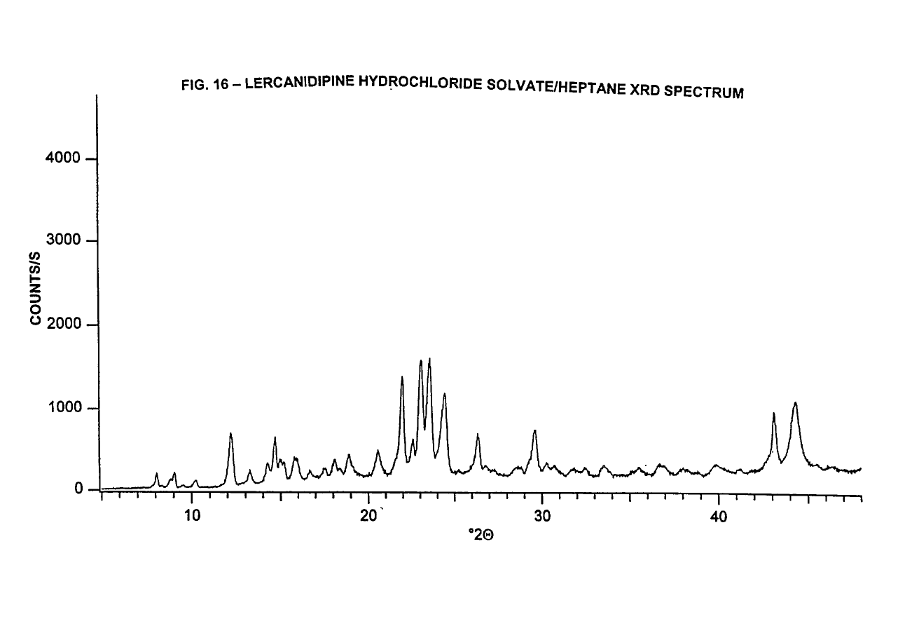

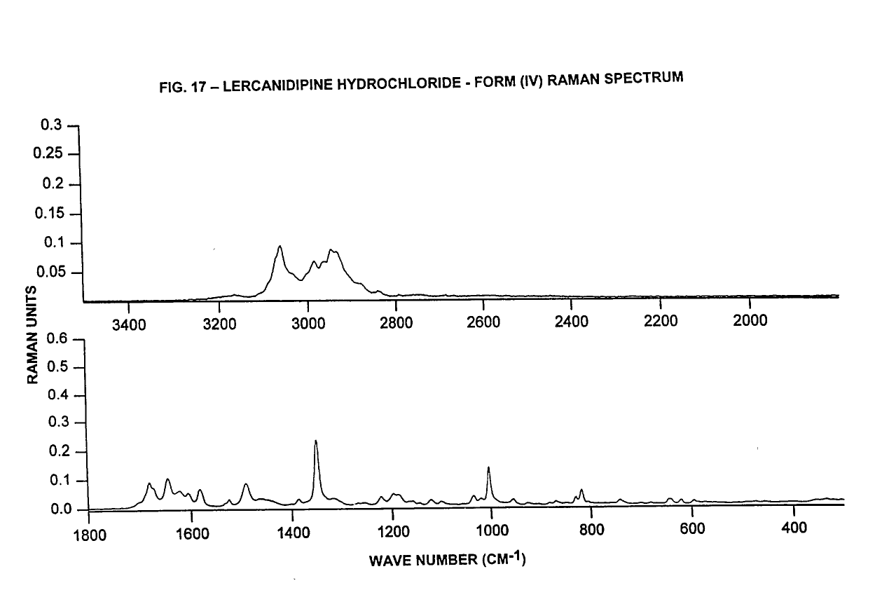

The solvate of lercanidipine hydrochloride with heptane showed an X-ray diffraction image, at wavelength Kα as expressed in Table 15 and shown in FIG. 16.

TABLE 15

| d (Å) | Relative intensity (%) | Angle (°2 θ) |
|---|---|---|
| 7.3 | 54 | 12.2 |
| 6.0 | 44 | 14.7 |
| 4.03 | 85 | 22.0 |
| 3.85 | 100 | 23.1 |
| 3.76 | 93 | 23.6 |
| 3.63 | 67 | 24.5 |
| 3.38 | 39 | 26.4 |
| 3.01 | 47 | 29.6 |

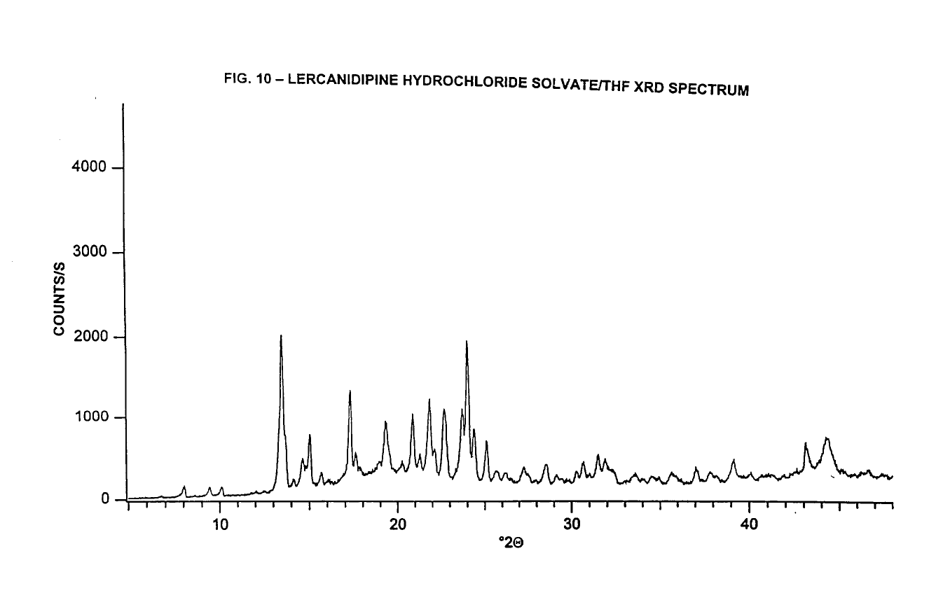

The solvate of lercanidipine hydrochloride with tetrahydrofuran showed an X-ray diffraction image, at wavelength Kα as expressed in Table 16 and shown in FIG. 10.

TABLE 16

| d (Å) | Relative intensity (%) | Angle (°2 θ) |
|---|---|---|
| 6.6 | 100 | 13.5 |
| 5.88 | 32 | 15.1 |
| 5.12 | 56 | 17.3 |
| 4.25 | 38 | 20.9 |
| 4.06 | 50 | 21.9 |
| 3.92 | 42 | 22.7 |
| 3.75 | 44 | 23.7 |
| 3.70 | 90 | 24.0 |
| 3.64 | 31 | 24.4 |

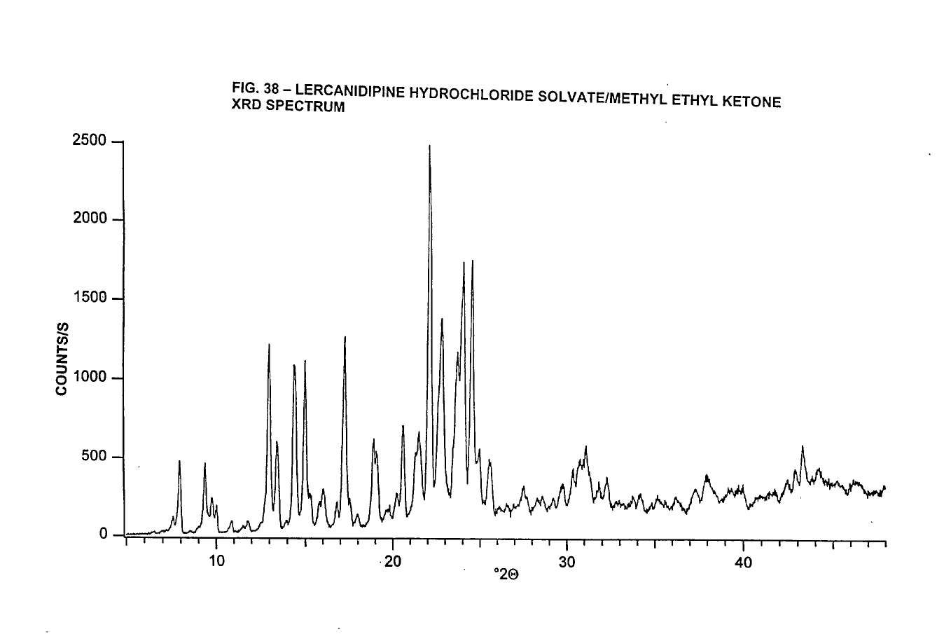

The solvate of lercanidipine hydrochloride with methyl ethyl ketone showed an X-ray diffraction image, at wavelength Kα as expressed in Table 17 and shown in FIG. 38.

TABLE 17

| d (Å) | Relative intensity (%) | Angle (°2 θ) |
|---|---|---|
| 6.8 | 50 | 13.1 |
| 6.1 | 43 | 14.5 |
| 5.87 | 47 | 15.1 |
| 5.10 | 53 | 17.4 |
| 3.99 | 100 | 22.2 |
| 3.87 | 48 | 22.9 |
| 3.74 | 36 | 23.8 |
| 3.69 | 65 | 24.1 |
| 3.61 | 70 | 24.6 |

### Example 36

Description of the Crystals and Their Thermal Characterization

### Example 36

### A Thermomicroscopic Analysis

A few mg of each sample were placed on a microscope slide provided with cover slip and placed on a Mettler model FP82 hotplate (Mettler, Volketswil, Switzerland) with a heating speed of 10° C./min, and analyzed with a Leitz Orthoplan Pol light microscope (Wild Leitz, Zurich, Switzerland) The sample was not hermetically sealed. The analysis provided the following results.

Solvate of Lercanidipine Hydrochloride with Methylene Chloride Prepared According to Example 1:The sample consisted of irregular striated birefringent crystals (examined with a crossed polarizer). The heating of the solvate resulted in the melting of the powder in a range between 138 and 150° C. No other transition phase was visible.

Lercanidipine hydrochloride crystalline Form (III) obtained as described in Example 2:The sample consisted of small and very small birefringent crystals (examined with a crossed polarizer) with an irregular shape and having breaks and cracks. The heating of the crystalline Form (III) resulted in a melting in a range of 137-150° C. No other transition phase was visible.

Solvate lercanidipine hydrochloride-anisole (b) form obtained as described in Example 3:The sample consisted of small birefringent cylinders (examined with a crossed polarizer), having breaks and cracks. No transition phase was observed up to the melting temperature of 144-146° C.

Solvate lercanidipine hydrochloride-ethyl acetate obtained as described in Example 4:The sample consisted of small birefringent cylinders (examined with a crossed polarizer), having breaks and cracks. Some small drops built up at 106° C. No transition phase was observed up to the melting temperature of 135-145° C.

Solvate lercanidipine hydrochloride-anisole (a) form obtained as described in Example 5:The sample consisted of birefringent crystals (examined with a crossed polarizer). Formation of microdrops together with the presence of several microcrystals was observed at 95° C.; no other transformation was seen by heating to melting at 188° C.

Solvate lercanidipine hydrochloride-terbutyl methyl ether obtained as described in Example 6:The sample consisted of non-birefringent crystals (examined with a crossed polarizer). Some small drops built up upon pressing the sample with a spatula. No transition phase was observed up to the melting temperature of 172-190° C.

Solvate lercanidipine hydrochloride-isopropanol (Example 7):The sample consisted of small birefringent cylinders (with a crossed polarizer) without breaks or cracks. From a range of 135-148° C. the crystals de-solvate and remained bathed in the liquid. The crystals melted at 177-200° C.

Solvate lercanidipine hydrochloride-2-butanol (Example 8):The sample consisted of birefringent cylinders (with a crossed polarizer) having several breaks and cracks. No transition phase was observed when the crystals were heated up to their melting temperature of 125-145° C.

Solvate lercanidipine hydrochloride-heptane (Example 9):The sample consisted of small irregular birefringent crystals (with a crossed polarizer). No transition phase was observed when the crystals were heated up to their melting point at 125-150° C.

Solvate lercanidipine hydrochloride-acetone (Example 10): The sample consisted of large irregular birefringent crystals (with a crossed polarizer). No transition phase was observed when the crystals were heated up to their melting temperature at 125-135° C.

Solvate lercanidipine hydrochloride-tetrahydrofuran (Example 11):The sample consisted of irregular crystals having breaks and cracks, which were birefringent (examined with a crossed polarizer). No transition phase was observed if the crystals were heated up to their melting point of 125-160° C.

Lercanidipine hydrochloride crystalline Form IV (Example 29):The sample consisted of large crystals having several breaks and cracks that were practically non birefringent (examined with a crossed polarizer). No transition phase was observed when the crystals were heated up to their melting temperature of 116-135° C. A few crystals kept their solid form and melted only at 195° C.

Solvate lercanidipine hydrochloride-methyl ethyl ketone (Example 34):The sample consisted of small cylinder-shaped birefringent crystals (with a crossed polarizer) having breaks and cracks. No transition phase was observed when the sample was heated up to the melting temperature (135-155° C.).

### Example 36B

### Thermogravimetric Analysis (TG and TGFTIR)

Each sample weighing 2 to 5 mg was placed in an aluminum crucible of an apparatus PERKIN ELMER TGS-2 Thermogravimetric System (Perkin-Elmer International, Inc., Rotkreuz, Switzerland) and heated in nitrogen stream at a rate of 10° C./min. The thermogravimetric analysis together with an IR analysis in Fourier transform was carried out according to the following operating modes. Each sample weighing 2 to 5 mg was placed in an aluminum crucible of an apparatus Netzsch Thermomicrobalance TG209 (Netzsch Gerätebau, Seib, Germany) coupled with a spectrometer in Fourier transform BRUKER FTIR Vector 22 (Spectrospin, Fällanden, Switzerland) and heated in nitrogen stream at a rate of 10° C./min.

The thermogravimetric analyses gave the following results:

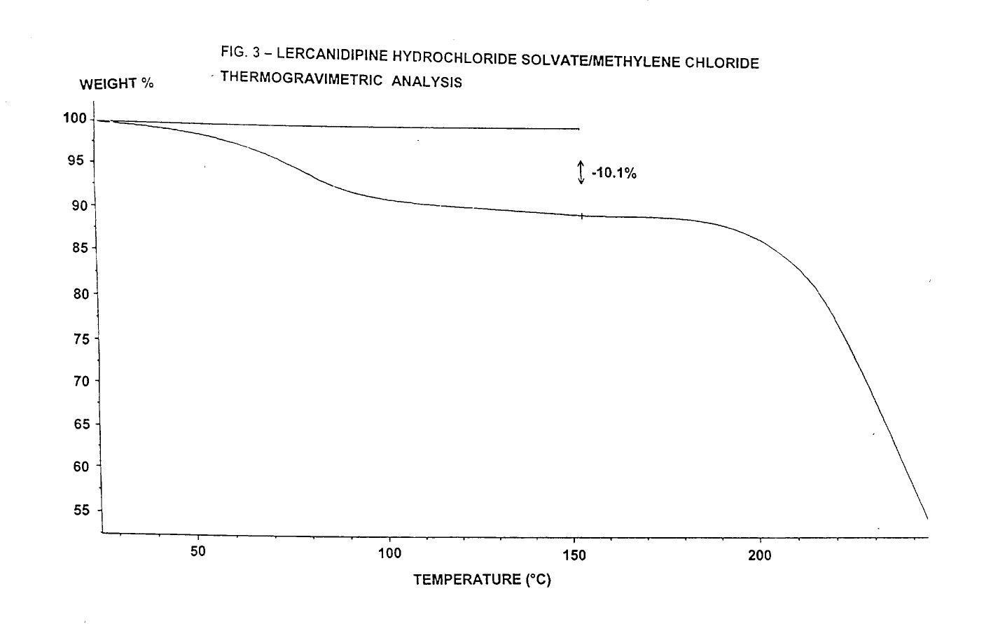

Solvate of lercanidipine hydrochloride with methylene chloride prepared according to Example 1:A weight loss of 10.1% was observed in the temperature range between 25 and 150° C. (FIG. 3).

The volatile compound was identified by the corresponding IR spectrum and was found to be methylene chloride. The stoichiometric compound monosolvate corresponded to a weight loss of 11.6%. Since methylene chloride has a high vapor pressure and since the sample already lost small amounts of dichloromethane at 25° C., it can be inferred that the product obtained in Example 1 corresponded to a solvate of lercanidipine hydrochloride with 1 molecule of methylene chloride.

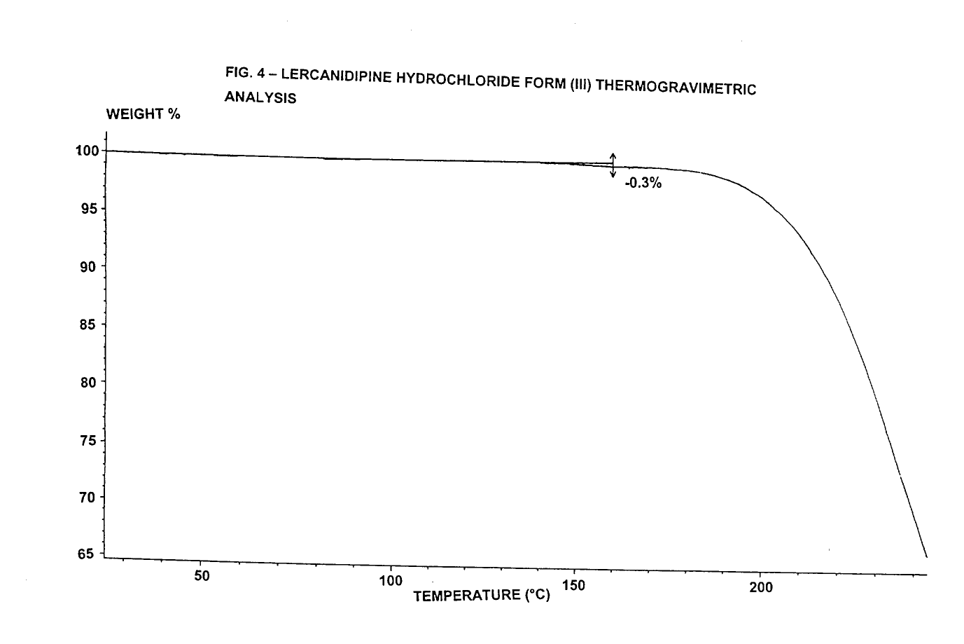

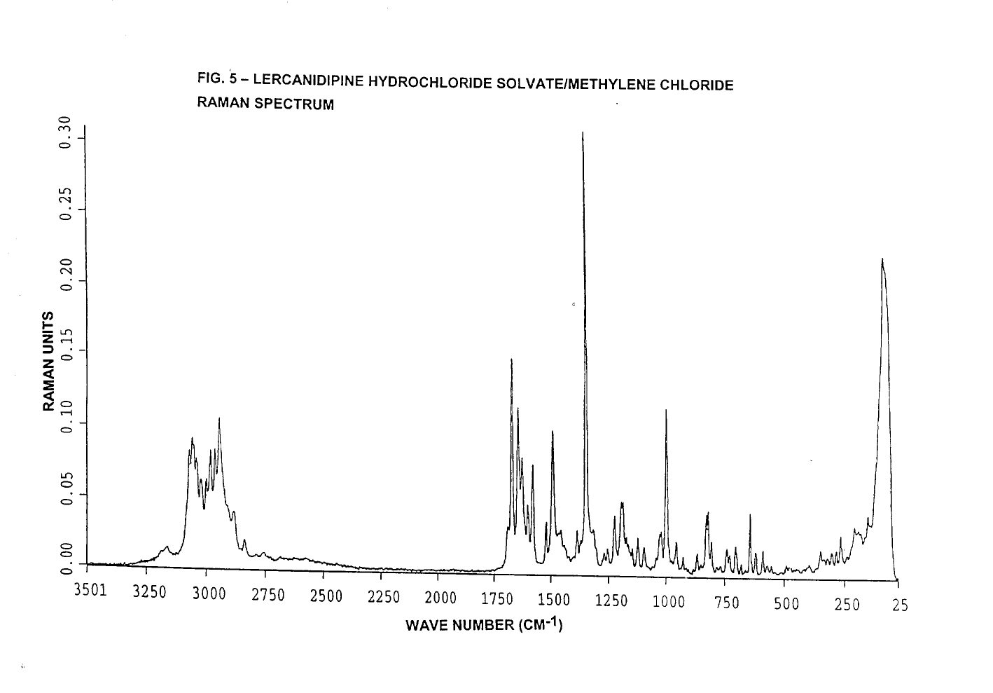

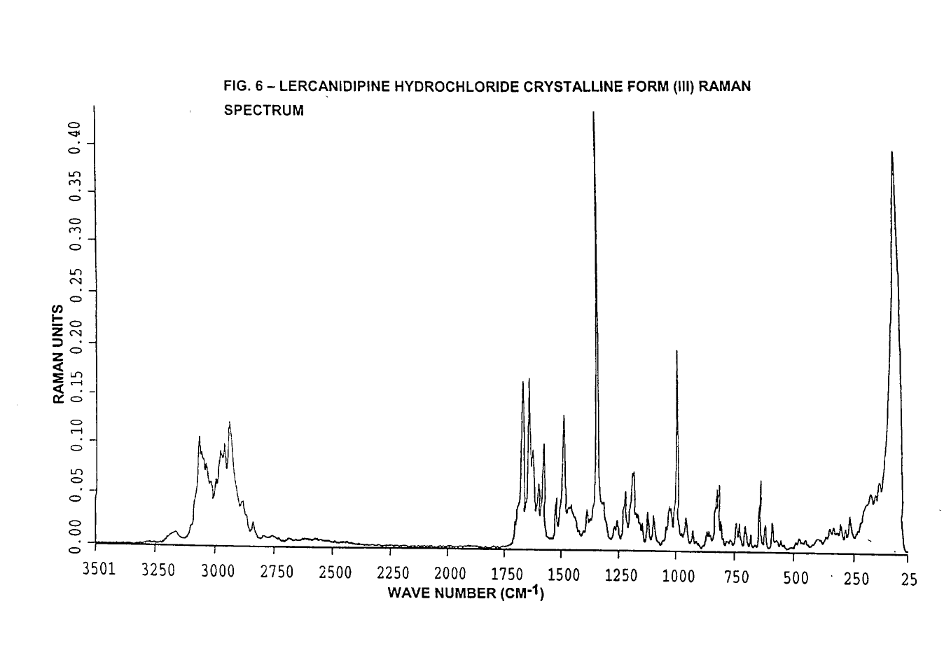

Lercanidipine hydrochloride crystalline Form (III) obtained as described in Example 2:A weight loss of 0.3% corresponding to the presence of dichloromethane, as identified by the corresponding IR spectrum, was observed in the temperature range 25-165° C. (See FIG. 4).

Solvate lercanidipine hydrochloride-anisole (b) form obtained as described in Example 3:A weight loss of 6.1% was observed in the range 25-170° C. (FIG. 27). Anisole was mainly present in the gas phase.

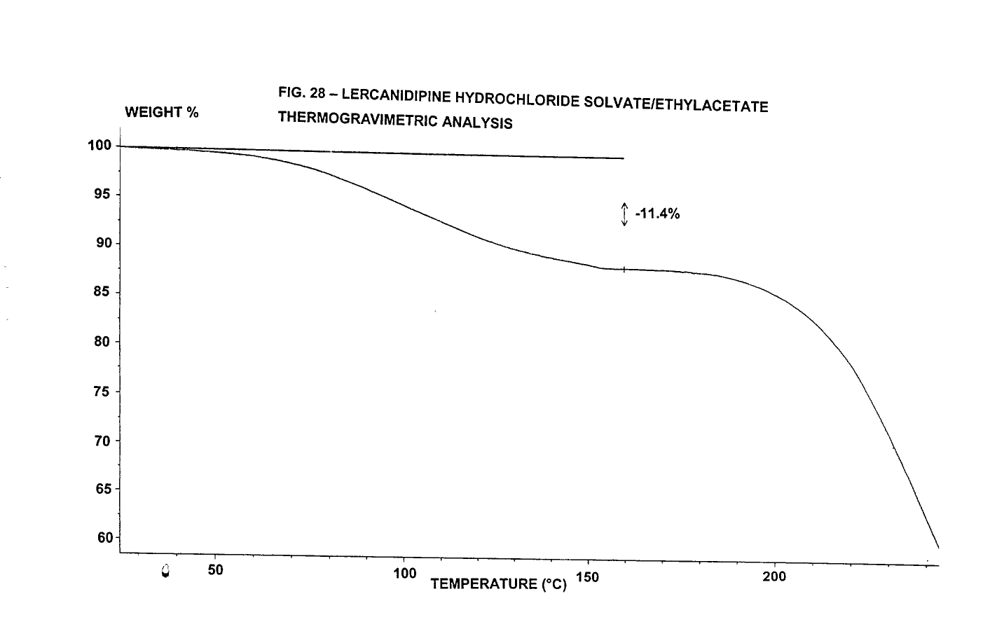

Solvate lercanidipine hydrochloride-ethyl acetate obtained as described in Example 4:A weight loss of 11.4% was observed in the temperature range between 25 and 160° C. (FIG. 28). The volatile compound, as identified by the IR spectrum, was found to be ethyl acetate.

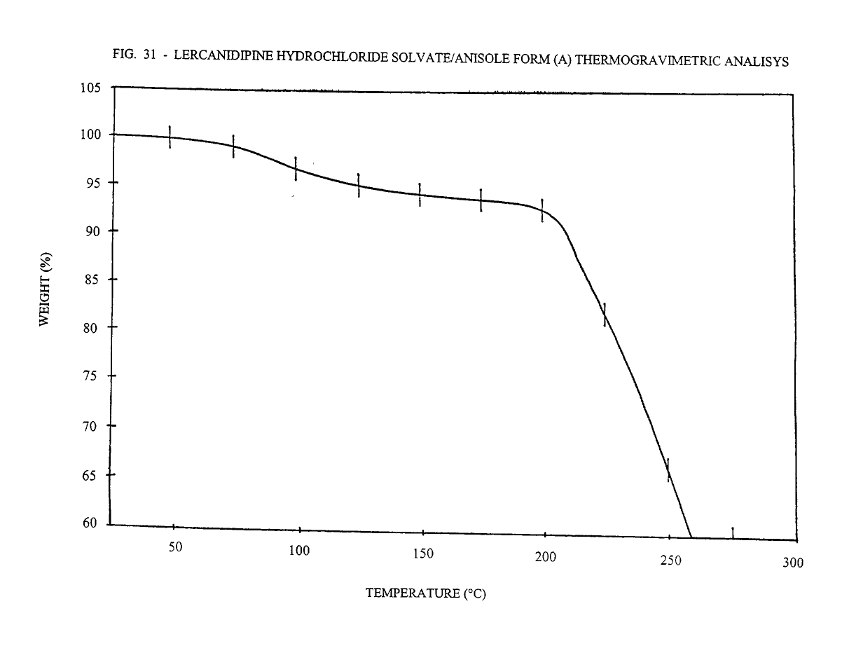

Solvate lercanidipine hydrochloride-anisole (a) form obtained as described in Example 5:A weight loss of 5.9% was observed in the temperature range between 25 and 175° C. (FIG. 31). The volatile compound was found to be anisole.

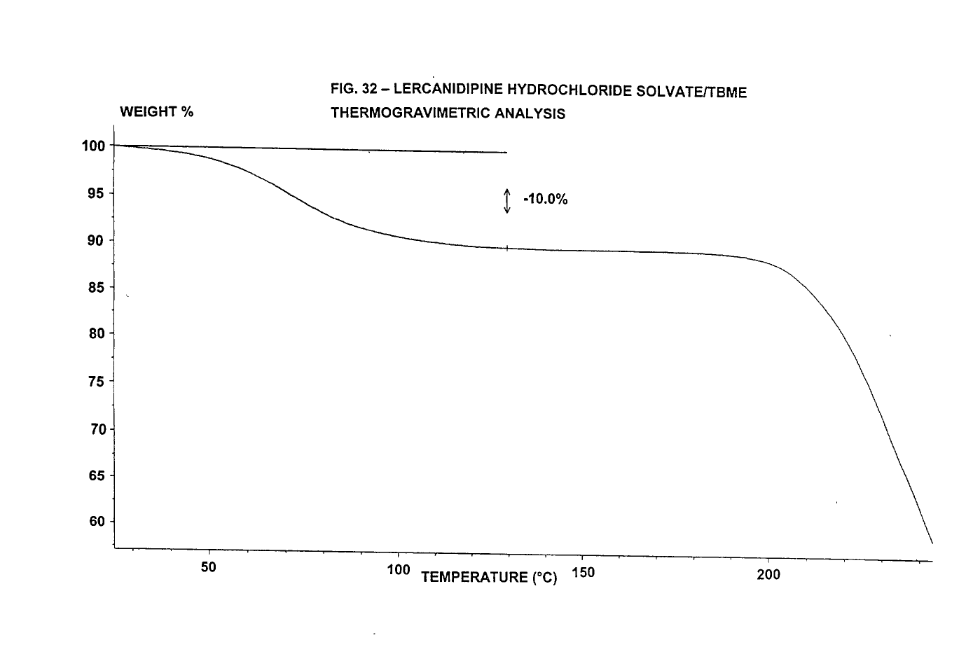

Solvate lercanidipine hydrochloride-terbutyl methyl ether obtained as described in Example 6:A weight loss of 10% was observed in the temperature range between 25 and 130° C. (FIG. 32). The volatile compound, as identified by the IR spectrum, was found to be terbutyl methyl ether. Degradation was observed at a temperature above 180° C. (only CO₂ is present).

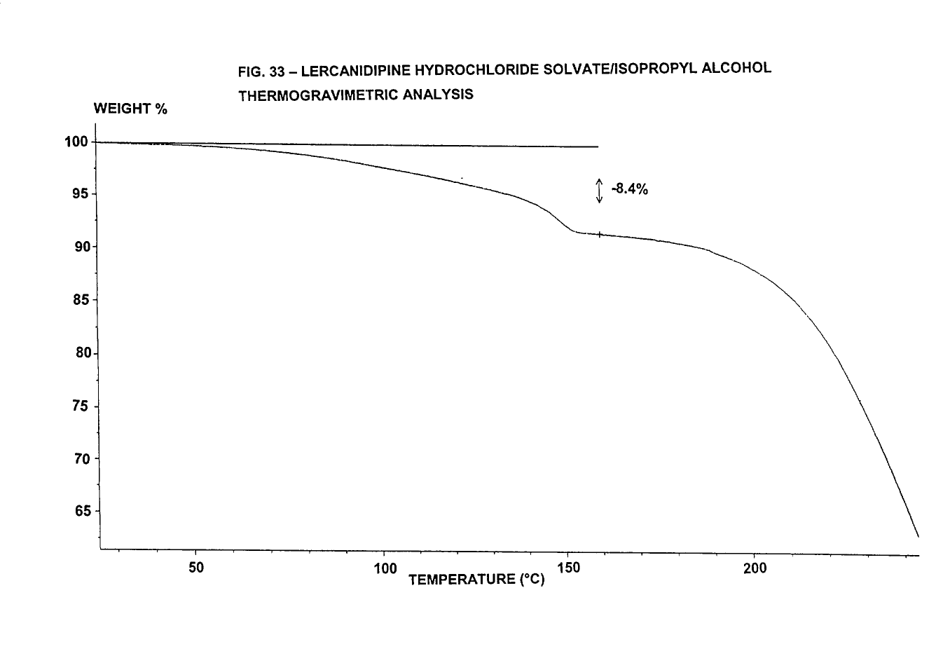

Solvate lercanidipine hydrochloride-isopropanol obtained as described in Example 7:A weight loss of 8.4% is observed in the temperature range between 25 and 160° C. (FIG. 33). The volatile component is found to be isopropanol.

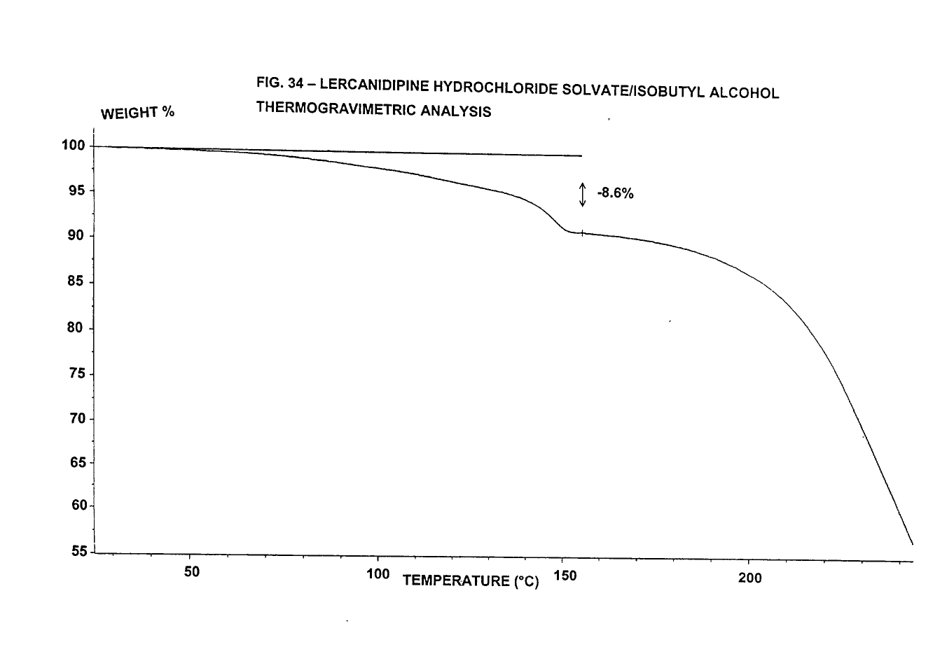

Solvate lercanidipine hydrochloride-2-butanol obtained as described in Example 8:A weight loss of 8.6% was observed in the temperature range 25-155° C. (FIG. 34). The volatile component was found to consist of 2-butanol.

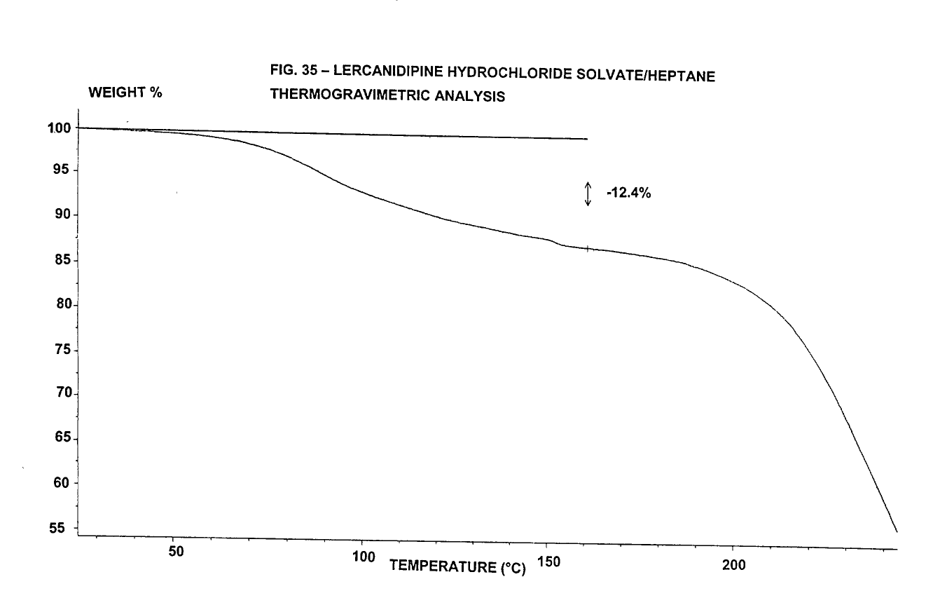

Solvate lercanidipine hydrochloride-heptane obtained as described in Example 9: A weight loss of 12.4% was observed in the temperature range 25-160° C. (FIG. 35).

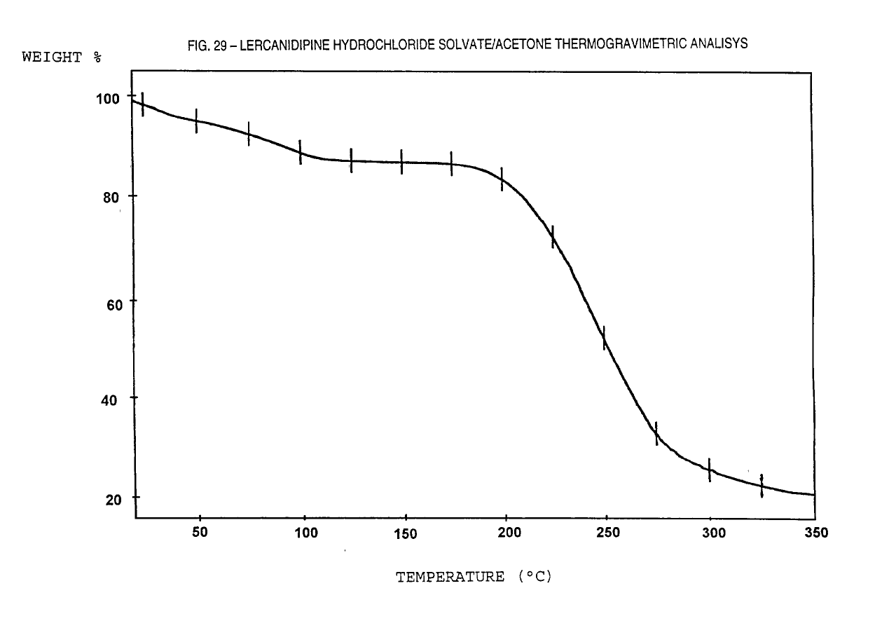

Solvate lercanidipine hydrochloride-acetone obtained as described in Example 10: A weight loss of 10.1% was observed in the temperature range 25-175° C. (FIG. 29).

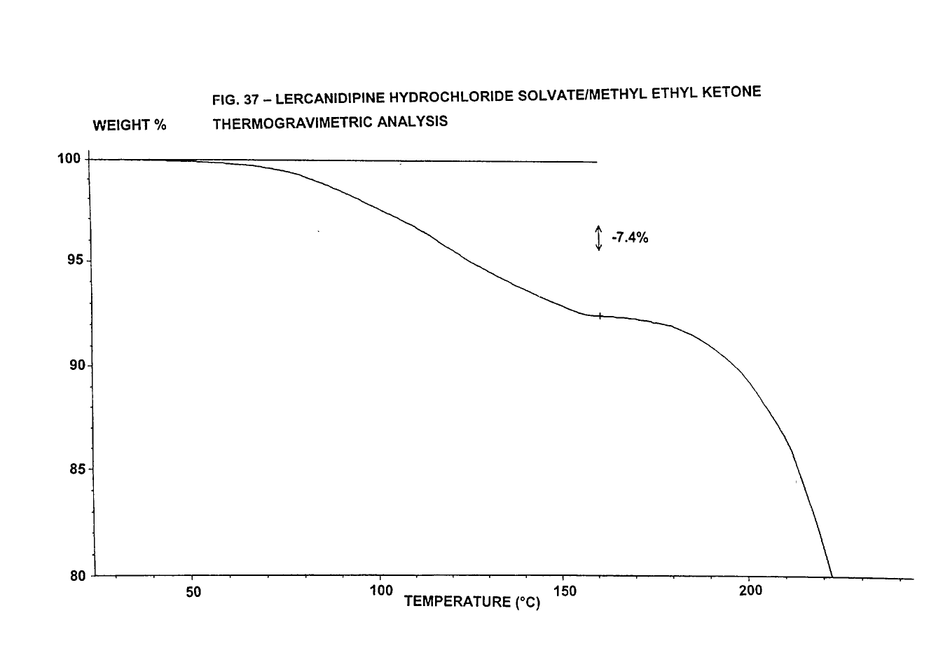

Solvate lercanidipine hydrochloride-methyl ethyl ketone obtained as described in Example 34:A weight loss of 7.4% was observed in the temperature range 25-160° C., (FIG. 37). The volatile compound identified was found to be methyl ethyl ketone.

Solvate lercanidipine hydrochloride-tetrahydrofuran (Example 11):A weight loss of 9.3% was observed at 25-180° C. The volatile component was found to be THF (FIG. 35).

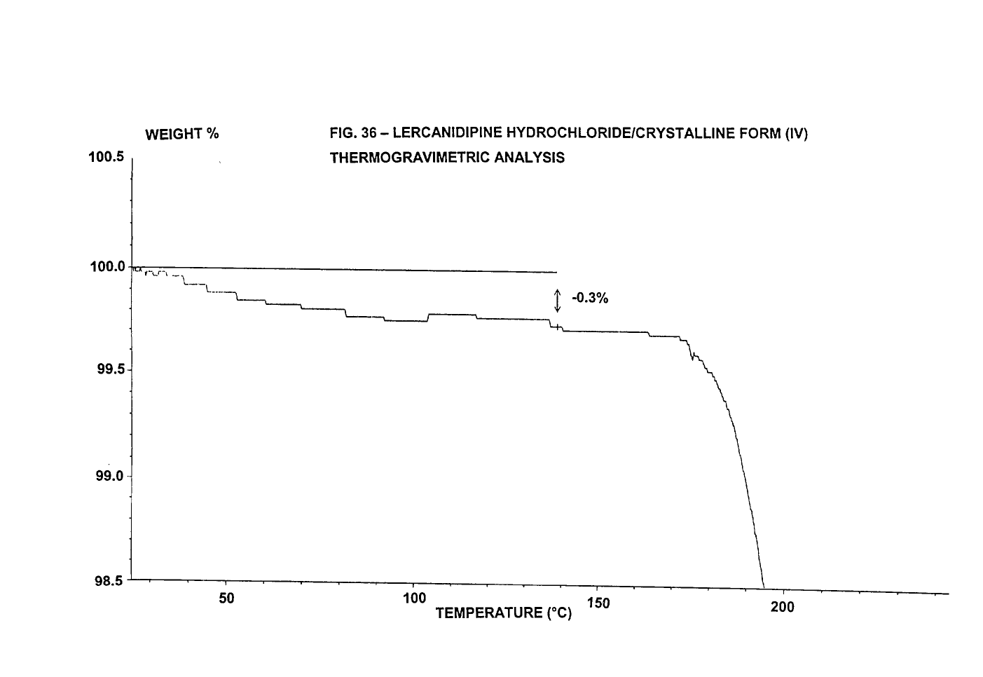

Lercanidipine hydrochloride Form (IV) (Example 29):A weight loss of 0.3% was observed between 25 and 140° C.; the volatile component was water (FIG. 36).

For some samples, mass loss did not correspond to stoichiometric values, which can be due to the presence of inclusion complexes.

The present invention is not to be limited in scope by the specific embodiments described herein. Various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

The disclosure of all patents, patent applications, publications, procedures, and the like that are cited throughout this application are hereby incorporated herein by reference in their entireties.

What is claimed is:

1. A solvate of lercanidipine hydrochloride comprising lercanidipine hydrochloride and an organic solvent, wherein the solvent is selected from the group consisting of methylene chloride, acetone, anisole, tetrahydrofuran, terbutyl methyl ether, isopropanol, 2-butanol, heptane, methyl ethyl ketone, and ethyl acetate.

2. The solvate according to claim 1, wherein when the solvent is methylene chloride, the lercanidipine hydrochloride-methylene chloride content is 1:1 (mole/mole) and the solvate has, at wavelength Kα, an X-ray diffraction image expressed by the following Table:

| d (Å) | Relative intensity (%) | Angle (° 2 θ) |
|---|---|---|
| 6.6 | 40 | 13.4 |
| 5.87 | 42 | 15.1 |
| 5.04 | 39 | 17.6 |
| 4.00 | 96 | 22.2 |
| 3.90 | 29 | 22.8 |
| 3.86 | 34 | 23.0 |
| 3.67 | 100 | 24.2 |
| 2.04 | 31 | 44.4 |

3. The solvate according to claim 1, wherein when the solvent is anisole, the lercanidipine hydrochloride-anisole content is 1:0.4 (mole/mole) and the solvate (a) form has, at wavelength Kα, an X-ray diffraction image expressed by the following Table:

| d (Å) | Relative intensity (%) | Angle (° 2 θ) |
|---|---|---|
| 17.4 | 62 | 5.1 |
| 7.6 | 34 | 11.6 |
| 5.71 | 43 | 15.5 |
| 5.57 | 58 | 15.9 |
| 4.99 | 47 | 17.7 |
| 4.62 | 40 | 19.2 |
| 4.44 | 29 | 20.0 |
| 4.28 | 98 | 20.8 |
| 4.04 | 100 | 22.0 |
| 3.19 | 43 | 27.9 |
| 2.92 | 36 | 30.6 |
| 2.86 | 42 | 31.3 |

4. The solvate according to claim 1, wherein when the solvent is anisole, the lercanidipine hydrochloride-anisole content is 1:0.4 (mole/mole) and the solvate (b ) form has, at wavelength Kα, an X-ray diffraction image expressed by the following Table:

| d (Å) | Relative intensity (%) | Angle (° 2 θ) |
|---|---|---|
| 6.9 | 49 | 12.8 |
| 6.7 | 63 | 13.3 |
| 5.82 | 86 | 15.2 |
| 5.27 | 41 | 16.8 |
| 5.15 | 53 | 17.2 |
| 4.00 | 47 | 22.2 |
| 3.89 | 46 | 22.8 |
| 3.66 | 100 | 24.3 |

5. The solvate according to claim 1, wherein when the solvent is acetone, the lercanidipine hydrochloride-acetone content is 1:1.2 (mole/mole) and the solvate has, at wavelength Kα, an X-ray diffraction image expressed by the following Table:

| d (Å) | Relative intensity (%) | Angle (° 2 θ) |
|---|---|---|
| 10.1 | 42 | 8.8 |
| 7.3 | 100 | 12.1 |
| 5.87 | 31 | 15.1 |
| 4.07 | 41 | 21.8 |
| 3.96 | 52 | 22.4 |
| 3.79 | 49 | 23.5 |
| 3.71 | 37 | 24.0 |
| 3.34 | 33 | 26.7 |

6. The solvate according to claim 1, wherein when the solvent is ethyl acetate, the lercanidipine hydrochloride-ethyl acetate content is 1:1 (mole/mole) and the solvate has, at wavelength Kα, an X-ray diffraction image expressed by the following Table:

| d (Å) | Relative intensity (%) | Angle (° 2 θ) |
|---|---|---|
| 6.9 | 100 | 12.8 |
| 6.3 | 29 | 14.0 |
| 5.80 | 45 | 15.3 |
| 5.65 | 31 | 15.7 |
| 5.43 | 44 | 16.3 |
| 4.74 | 53 | 18.7 |
| 4.53 | 49 | 19.6 |
| 4.00 | 84 | 22.2 |
| 3.91 | 91 | 22.7 |
| 3.67 | 77 | 24.2 |
| 3.60 | 34 | 24.7 |
| 3.53 | 34 | 25.2 |
| 3.49 | 43 | 25.5 |

7. The solvate according to claim 1, wherein when the solvent is terbutyl methyl ether, the lercanidipine hydrochloride-terbutyl methyl ether content is 1:0.8 (mole/mole) and the solvate has, at wavelength Kα, an X-ray diffraction image expressed by the following Table:

| d (Å) | Relative intensity (%) | Angle (° 2 θ) |
|---|---|---|
| 6.2 | 77 | 14.2 |
| 4.88 | 29 | 18.2 |
| 4.52 | 64 | 19.6 |
| 4.02 | 48 | 22.1 |
| 3.93 | 100 | 22.6 |
| 3.43 | 46 | 26.0 |

8. The solvate according to claim 1, wherein when the solvent is isopropanol, the lercanidipine hydrochloride-isopropanol content is 1:1 (mole/mole) and the solvate has, at wavelength Kα, an X-ray diffraction image expressed by the following Table:

| d (Å) | Relative intensity (%) | Angle (° 2 θ) |
|---|---|---|
| 6.6 | 35 | 13.5 |
| 5.85 | 48 | 15.1 |
| 5.06 | 41 | 17.5 |
| 4.04 | 64 | 22.0 |
| 3.90 | 39 | 22.8 |
| 3.72 | 37 | 23.9 |
| 3.67 | 100 | 24.2 |

9. The solvate according to claim 1, wherein when the solvent is 2-butanol, the lercanidipine hydrochloride-2-butanol content is 1:0.8 (mole/mole) and the solvate has, at wavelength Kα, an X-ray diffraction image expressed by the following Table:

| d (Å) | Relative intensity (%) | Angle (° 2 θ) |
|---|---|---|
| 6.8 | 34 | 13.1 |
| 5.86 | 36 | 15.1 |
| 5.13 | 42 | 17.3 |
| 4.03 | 51 | 22.0 |
| 3.90 | 36 | 22.8 |
| 3.67 | 100 | 24.2 |

10. The solvate according to claim 1, wherein when the solvent is heptane, the lercanidipine hydrochloride-heptane content is 1:0.9 (mole/mole) and the solvate has, at wavelength Kα, an X-ray diffraction image expressed by the following Table:

| d (Å) | Relative intensity (%) | Angle (° 2 θ) |
|---|---|---|
| 7.3 | 54 | 12.2 |
| 6.0 | 44 | 14.7 |
| 4.03 | 85 | 22.0 |
| 3.85 | 100 | 23.1 |
| 3.76 | 93 | 23.6 |
| 3.63 | 67 | 24.5 |
| 3.38 | 39 | 26.4 |
| 3.01 | 47 | 29.6 |

11. The solvate according to claim 1, wherein when the solvent is methyl ethyl ketone, the lercanidipine hydrochloride-methyl ethyl ketone content is 1:0.7 (mole/mole) and the solvate has, at wavelength Kα, an X-ray diffraction image expressed by the following Table:

| d (Å) | Relative intensity (%) | Angle (° 2 θ) |
|---|---|---|
| 6.8 | 50 | 13.1 |
| 6.1 | 43 | 14.5 |
| 5.87 | 47 | 15.1 |
| 5.10 | 53 | 17.4 |
| 3.99 | 100 | 22.2 |
| 3.87 | 48 | 22.9 |
| 3.74 | 36 | 23.8 |
| 3.69 | 65 | 24.1 |
| 3.61 | 70 | 24.6 |

12. The solvate according to claim 1, wherein when the solvent is tetrahydrofuran, the lercanidipine hydrochloride-tetrahydrofuran content is 1:0.9 (mole/mole) and the solvate has, at wavelength Kα, an X-ray diffraction image expressed by the following Table:

| d (Å) | Relative intensity (%) | Angle (° 2 θ) |
|---|---|---|
| 6.6 | 100 | 13.5 |
| 5.88 | 32 | 15.1 |
| 5.12 | 56 | 17.3 |
| 4.25 | 38 | 20.9 |
| 4.06 | 50 | 21.9 |
| 3.92 | 42 | 22.7 |
| 3.75 | 44 | 23.7 |
| 3.70 | 90 | 24.0 |
| 3.64 | 31 | 24.4 |

13. Isolated lercanidipine hydrochloride crystalline form (III), having an X-ray diffraction image, at wavelength Kα, expressed by the following Table:

| d (Å) | Relative intensity (%) | Angle (° 2 θ) |
|---|---|---|
| 11.5 | 39 | 7.7 |
| 9.1 | 38 | 9.7 |
| 9.0 | 37 | 9.8 |
| 8.0 | 50 | 11.0 |
| 6.6 | 48 | 13.5 |
| 5.58 | 57 | 15.9 |
| 5.49 | 34 | 16.1 |
| 5.13 | 43 | 17.3 |
| 4.09 | 63 | 21.7 |
| 3.92 | 43 | 22.7 |
| 3.72 | 100 | 23.9 |
| 3.60 | 85 | 24.7 |
| 3.47 | 31 | 25.6 |

14. The lercanidipine hydrochloride crystalline form of claim 13 characterized in that it is in micronized form.

15. The lercanidipine hydrochloride crystalline form of claim 14 characterized in that the average size of said micronized form is of D(50%) 2-8 μm, D(90%)<15 μm.

16. Isolated lercanidipine hydrochloride crystalline form (IV), having an X-ray diffraction image, at wavelength Kα, expressed by the following Table:

| d (Å) | Relative intensity (%) | Angle (° 2 θ) |
|---|---|---|
| 7.9 | 71 | 11.2 |
| 6.9 | 53 | 12.7 |
| 5.21 | 57 | 17.0 |
| 5.13 | 46 | 17.3 |
| 4.73 | 66 | 18.8 |
| 4.69 | 95 | 18.9 |
| 4.53 | 53 | 19.6 |
| 4.40 | 81 | 20.2 |
| 4.34 | 43 | 20.4 |
| 3.99 | 44 | 22.2 |
| 3.89 | 52 | 22.8 |
| 3.77 | 100 | 23.6 |
| 3.69 | 35 | 24.1 |

17. The lercanidipine hydrochloride crystalline form of claim 16 characterized in that it is in micronized form.

18. The lercanidipine hydrochloride crystalline form of claim 17 characterized in that the average size of said micronized form is of D(50%) 2-8 μm, D(90%)<15 μm.

19. An antihypertensive pharmaceutical composition comprising (1) a first lercanipidine form selected from the group consisting of lercanidipine hydrochloride crystalline Form (III), lercanidipine hydrochloride crystalline Form (IV) and combinations thereof, each crystalline form having a predetermined content; and (2) at least one component selected from the group consisting of a pharmaceutically acceptable carrier or diluent, a flavorant, a sweetener, a preservative, a dye, a binder, a suspending agent, a dispersing agent, a colorant, a disintegrant, an excipient, a lubricant, a plasticizer, and an edible oil.

20. The unit dosage form of claim 19 further comprising a second lercanidipine form that is not a predetermined amount of lercanidipine hydrochloride crystalline Form (III) or lercanidipine hydrochloride crystalline Form (IV) or combination thereof.

21. The unit dosage form of claim 20 wherein said second lercanidipine form is selected from one or more of the group consisting of lercanidipine hydrochloride crystalline Form (I), lercanidipine hydrochloride crystalline Form (II) and amorphous lercanidipine.

22. The unit dosage form of claim 21 wherein said second lercanidipine form is selected from one or more of the group consisting of a predetermined amount of lercanidipine hydrochloride crystalline Form (I), a predetermined amount of lercanidipine hydrochloride crystalline Form (II) and amorphous lercanidipine.

23. The unit dosage form of claim 22 wherein said second lercanidipine form is selected from one or more of the group consisting of a predetermined amount of lercanidipine hydrochloride crystalline Form (I) and a predetermined amount of lercanidipine hydrochloride crystalline Form (II).

24. A unit dosage form comprising the antihypertensive pharmaceutical composition of any one of claims 19-23.

25. The unit dosage form of claim 24, wherein the form comprises 0.1 to 400 mg lercanidipine hydrochloride.

26. The unit dosage form of claim 25, wherein the form comprises 1 to 200 mg lercanidipine hydrochloride.

27. The unit dosage form of claim 26, wherein the form comprises 5 to 40 mg lercanidipine hydrochloride.

28. The unit dosage form of claim 27, wherein the form comprises not below 0.1% by weight of lercanidipine hydrochloride, based on the total weight of the form.

29. The unit dosage form of claim 28, wherein the form comprises between 0.5 and 30% by weight of lercanidipine hydrochloride based on the total weight of the form.

30. A method of making a lercanidipine hydrochloride solvate comprising:
(i) mixing lercanidipine crystalline Form (I), lercanidipine crystalline Form (III), crude lercanidipine form (A), crude lercanidipine form (B), crude lercanidipine form (C) or a lercanidipine hydrochloride-methylene chloride solvate with an organic solvent to form a solvate of lercanidipine hydrochoride and said organic solvent; and
(ii) isolating the solvate formed in step (i),
with the proviso that step (i) does not consist of mixing a lercanidipine hydrochloride-methylene chloride solvate with the organic solvent methylene chloride.

31. The method of claim 30 wherein step (i) is performed at a temperature of about 20-50° C.

32. The method of claim 31 wherein step (i) comprises mixing at room temperature.

33. The method of claim 30 wherein step (i) comprises stirring for 114-420 hours.

34. The method of claim 30, wherein step (i) comprises at least one thermal cycle, wherein the cycle is comprised of a first cooling step, a heating step and a second cooling step.

35. The method of claim 34, wherein the first and second cooling steps are 25° C. and the heating step is 35° C.

36. The method of claim 35, wherein each step is 3 hours.

37. The method of claim 34, wherein the cycle is repeated between 10 and 20 times.

38. The method of claim 37, wherein at the completion of the cycles, the mixture is stirred at 25° C. for 24-240 hours.

39. The method of claim 30 wherein said organic solvent is selected from the group consisting of methylene chloride, methyl ethyl, acetone, anisole, ethyl acetate, tetrahydrofuran, terbutyl methyl ether, isopropanol, 2-butanol and heptane.

40. The method of claim 31 wherein the solvate formed in step (i) is selected from the group consisting of lercanidipine hydrochloride-methylene chloride, lercanidipine hydrochloride-methyl ethyl ketone, lercanidipine hydrochloride-acetone, lercanidipine hydrochloride-anisole form (a), lercanidipine hydrochloride-anisole form (b),lercanidipine hydrochloride-ethyl acetate, lercanidipine hydrochloride-tetrahydrofuran, lercanidipine hydrochloride-terbutyl methyl ether, lercanidipine hydrochloride-isopropanol, lercanidipine hydrochloride-2-butanol and lercanidipine hydrochloride-heptane.

41. The method of claim 30 wherein:
step (i) comprises mixing lercanidipine hydrochloride crystalline Form (I), lercandipine crude form (A), lercanidipine crude form (B) or lercanidipine crude form (C) with methylene chloride to form said lercanidipine hydrochloride-methylene chloride solvate; and
step (ii) comprises isolating the lercanidipine hydrochloride-methylene chloride solvate formed in step (i).

42. The method of claim 41 wherein step (i) comprises stirring at a temperature between 20 and 50° C.

43. The method of claim 42, wherein step (i) is performed at room temperature.

44. The method of claim 41 wherein step (ii) comprises filtering the solvate formed in step (i).

45. The method of claim 30 wherein:
step (i) comprises mixing a lercanidipine hydrochloride-methylene chloride solvate with an organic solvent selected from the group consisting of acetone, anisole, ethyl acetate, tetrahydrofuran, terbutyl methyl ether, isopropanol, 2-butanol and heptane; and
step (ii) comprises isolating the solvate produced in step (i).

46. The method of claim 45 wherein said lercanidipine hydrochloride-methylene chloride solvate is produced by the method of one of claims 41-44.

47. The method of claim 45 wherein step (i) is performed at a temperature between about 20-50° C.

48. The method of claim 45 wherein step (i) is performed for about 116-420 hours.

49. The method of claim 45 wherein step (ii) comprises filtering the solvate formed in step (i).

50. The method of claim 30 wherein
step (i) comprises mixing lercanidipine crystalline form (III) with a solvent selected from the group consisting of anisole, ethyl acetate, tetrahydrofuran, terbutyl methyl ether and acetone; and
step (ii) comprises isolating the solvate formed in step (i).

51. The method of claim 50 wherein step (i) is performed at a temperature between about 20-50° C.

52. The method of claim 50 wherein step (i) is performed for about 116-420 hours.

53. The method of claim 50 wherein step (ii) comprises filtering the solvate formed in step (i).

54. The method of claim 30 wherein
step (i) comprises mixing lercanidipine crystalline form (III) with a solvent selected from the group consisting of anisole, ethyl acetate, tetrahydrofuran, terbutyl methyl ether and acetone; and
step (ii) comprises isolating the solvate formed in step (i).

55. The method of claim 54 wherein step (i) is performed at a temperature between about 20-50° C.

56. The method of claim 54 wherein step (i) is performed for about 116-420 hours.

57. The method of claim 54 wherein step (ii) comprises filtering the solvate formed in step (i).

58. The method of claim 30 wherein
step (i) comprises mixing lercanidipine crude form (A) or crude form (B) with a solvent selected from the group consisting of anisole, ethyl acetate, tetrahydrofuran, terbutyl methyl ether, acetone and methylene chloride; and
step (ii) comprises isolating the solvate formed in step (i).

59. The method of claim 58 wherein step (i) is performed at a temperature between about 20-50° C.

60. The method of claim 58 wherein step (i) is performed for about 116-420 hours.

61. The method of claim 58 wherein step (ii) comprises filtering the solvate formed in step (i).

62. A method for preparing lercanidipine hydrochloride crystalline Form (III), comprising the removal of solvent from a lercanidipine hydrochloride solvate by evaporation under vacuum or in a nitrogen stream to form said crystalline Form (III).

63. The method of claim 62 wherein the solvent is selected from the group consisting of methylene chloride, tetrahydrofuran, heptane, anisole, ethyl acetate, isopropanol and 2-butanol.

64. The method of claim 63 wherein the acetone is removed in a vacuum of about 1-0.01 mbar for about 20-30 hours at a temperature of about 50-80° C.

65. The method of claim 63 wherein the solvent is removed from a solvate selected from the group consisting of:
lercanidipine hydrochloride with methylene chloride having an X-ray diffraction image, at wavelength Kα as expressed in Table 7;
lercanidipine hydrochloride with anisole (a) form having an X-ray diffraction image, at wavelength Kα as expressed in Table 8;
lercanidipine hydrochloride with anisole (b) form having an X-ray diffraction image, at wavelength Kα expressed in Table 9;
lercanidipine hydrochloride with ethyl acetate having an X-ray diffraction image, at wavelength Kα as expressed in Table 11;
lercanidipine hydrochloride with isopropanol having an X-ray diffraction image, at wavelength Kα as expressed in Table 13;
lercanidipine hydrochloride with 2-butanol having an X-ray diffraction image, at wavelength Kα the image as expressed in Table 14;
lercanidipine hydrochloride with heptane having an X-ray diffraction image, at wavelength Kα as expressed in Table 15; and
lercanidipine hydrochloride with tetrahydrofuran having an X-ray diffraction image, at wavelength Kα as expressed in Table 16.

66. The method of claim 63, wherein the method is performed at room temperature.

67. The method of claim 63, wherein the method further comprises at least one thermal cycle, wherein the cycle is comprises of a first cooling step, a heating step, and a second cooling step.

68. The method of claim 67, wherein the first and second cooling steps are 25° C. and the heating step is 35° C.

69. The method of claim 68, wherein each step is 3 hours.

70. The method of claim 67, wherein the cycle is repeated between 10 and 20 times.

71. The method of claim 70, wherein at the completion of the cycles, the mixture is stirred at 25° C. for 24-240 hours.

72. A method for preparing lercanidipine hydrochloride crystalline Form (IV), comprising removal of acetone from a lercanidipine hydrochloride-acetone solvate by evaporation under vacuum or in a nitrogen stream.

73. The method of claim 72 wherein the acetone is removed in a vacuum of about 1-0.01 mbarr for about 20-30 hours at a temperature of about 50-80° C.

74. A method for preparing a solvate lercanidipine hydrochloride-methyl ethyl ketone, comprising the steps of:
(i) mixing lercanidipine hydrochloride crystalline Form (I) with methyl ethyl ketone to obtain a solution;
(ii) optionally cooling the solution and continue stirring to produce the solvate lercanidipine hydrochloride-methyl ethyl ketone, and
(iii) isolating the solvate of step (ii).

75. The method of claim 74 wherein the solution of step (i) comprises 0-5% (v/v) water.

76. The method of claim 74 wherein step (i) is performed at 80° C.

77. The method of claim 74 wherein step (ii) comprises cooling to room temperature and stirring.

78. The method of claim 74 wherein step (ii) comprises stirring the solution at room temperature for two days.

79. The method of claim 74 wherein step (iii) comprises filtering.

80. The method of claim 74 wherein step (iii) comprises drying the solvate from step (ii) in an oven at about 60° C. for 24 hours.

81. A method of treating a subject with arterial hypertension, the method comprising administering a therapeutically effective amount of lercanidipine hydrochloride crystalline Form (III), lercanidipine hydrochloride crystalline form (IV), or combinations thereof to a subject in need of such treatment.

82. A method of treating or preventing atherosclerotic lesions in arteries in a subject, which comprises administering a herapeutically effective amount of lercanidipine hydrochloride crystalline Form (III), lercanidipine hydrochloride crystalline Form (IV), or combinations thereof having a predetermined content in each of said Form (III) and (IV) to a subject in need of such treatment.

83. A method of treating or preventing heart failure in a subject, which comprises administering a therapeutically effective amount of lercanidipine hydrochloride crystalline Form (III), lercanidipine hydrochloride crystalline Form (IV), or combinations thereof having a predetermined content in each of said Form (III) and (IV) to a subject in need of such treatment.

84. The method of any one of claim 81-83 wherein said subject is a mammal.

85. The method of claim 84 wherein said subject is a human.

86. An antihypertensive composition comprising predetermined amounts of lercanidipine hydrochloride crystalline Form (III) and lercanidipine hydrochloride crystalline Form (IV).

87. The antihypertensive composition of claim 86 wherein the ratio of Form (III):Form (IV) is between about 1:9 to 9:1.

88. The antihypertensive composition of claim 87 wherein the ration of Form (III):Form (IV) is selected from the group consisting of 9:1, 7:3, 1:1, 3:7 and 1:9.
